(19) United States
(12) Patent Application Publication
(10) Pub. No.: US 2003/0064025 A1
(43) Pub. Date: Apr. 3, 2003

(54) Imaging systems for in vivo protocols

(76) Inventors: Xiaoming Yang, Baltimore, MD (US); Ananda Kumar, Baltimore, MD (US); Hunter H. Chen, Baltimore, MD (US)

Correspondence Address: DIKE, BRONSTEIN, ROBERTS AND CUSHMAN,, INTELLECTUAL PROPERTY PRACTICE GROUP, EDWARDS & ANGELL, LLP., P.O. BOX 9169, BOSTON, MA, 02209, US

(21) Appl. No.: 10/117,432

(22) Filed: Apr. 5, 2002

Related U.S. Application Data
(60) Provisional application No. 60/281,565, filed on Apr. 5, 2001.

(51) Int. Cl. A61K 49/00; A61B 10/00; G06F 19/00; G01N 33/48; G01N 33/50

(52) U.S. Cl. 424/9.6; 702/19; 600/300

(57) ABSTRACT

New digital optical imaging techniques are provided which can enable intravascular, external, or minimally-invasive transpercutaneous tracking of vascular gene expression and diagnosis of a variety of disorders and diseases including atherosclerosis. Preferred optical imaging systems for use in the invention can detect extrinsic/intrinsic fluorescent/luminous signals emitted from deep-seated vessels.

This application claims the benefit of U.S. Provisional Application No. 60/281,565, filed Apr. 5, 2001, which is incorporated by reference herein in its entirety.

### BACKGROUND

1. Field of The Invention

The invention provides, inter alia, optical imaging methods for in vivo monitoring of vascular gene expression. Preferred methods of the invention comprise intravascularly administering to a patient a composition having a detectable component, and detecting the administered composition with use of a digital optical imaging system.

2. Background

Atherosclerotic cardiovascular disease remains the leading cause of mortality in the United States. Gene therapy is a rapidly expanding field with great potential for the treatment of atherosclerotic cardiovascular diseases. Several genes, such as vascular endothelial growth factor (VEGF), have been shown to be useful for preventing acute thrombosis, blocking post-angioplasty restenosis, and stimulating growth of new blood vessels (angiogenesis). However, precise monitoring of gene expression within targeted atherosclerotic plaques is a challenging task. To date, most investigations about imaging of gene therapy have been primarily focused on non-cardiovascular systems, and no in vivo imaging modalities are currently available for vascular gene therapy.

The emergence of a viable marker, green fluorescent protein (GFP), has opened the door for the convenient use of intact living cells and organisms as experimental systems in fields ranging from cell biology to biomedicine. GFP, which fluoresces autonomously in nearly all cells tested, has been increasingly used as a convenient and sensitive reporter of transgene expression that can be detected by fluorescence microscopy or flow cytometry. Biologically, GFP acts to shift the color of bioluminescence from blue to green in luminous coelenterates (jellyfish, hydroids, sea pansies, and sea pens) and to increase the quantum yield of light emission. This fluorescence can be visualized directly on culture plates upon illumination with either blue- or long-wave ultraviolet (UV) light. Any of the vectors designed for protein expression can be used to make constructs to express GFP in different cells or organisms, either alone or as a fusion protein. The expression of GFP within an organism produces an intrinsic fluorescence that colors normal cellular processes, and high-resolution optical techniques can be used to monitor the dynamic activities of these living cells.

Although a non-invasive imaging method would be ideal for tracking gene therapy in the vasculature, many challenges and impracticalities remain. The light scattering by molecular particles in a tissue and the pigments of a tissue can attenuate the intensity of light delivered and received; thus quantitative fluorescence measurements cannot be obtained without some type of empirical calibration. Deep-seated arteries such as the renal arteries could not be optically probed non-invasively.

Gene therapy is an exciting frontier in medicine today. Our current knowledge about the biodistribution and/or in vivo pharmacokinetics of gene therapy is incomplete and relies mainly on staining of biopsied or post-mortem tissues. A need, therefore, exists to monitor and detect the location of gene therapeutic agents. Preferably, this need is met with a minimal invasive or non-invasive means.

### SUMMARY OF THE INVENTION

The present invention provides for the digital optical imaging of gene expression from the vasculature. Such application of optical imaging technology to vascular gene therapy can provide substantial advantages, including: 1) portability for bedside or operating room monitoring; 2) lack of exposure to ionizing radiation; 3) relatively low cost; and 4) the ability to directly detect commercially-available imaging markers, such as biological fluorescent/luminous materials used for gene therapies. In addition, using the digital optical imaging system, the invention provides for the examination of the internal elastic lamina (IEL) by detecting the autofluorescence emitted from the fiber component of the vessel wall. This is of important clinical significance.

Preferred methods of the invention include detecting intravascularly administered molecules by administering to a patient (mammal, particularly human) a composition having a detectable component, and detecting the administered composition with use of a digital imaging system. The administered composition may be detectable by a variety of means with fluorescence (luminescence) being generally preferred. Suitably, after administration, the composition is activated, e.g. exposed to activating radiation, to provide detection such as fluorescence.

In preferred aspects, the invention provides for three distinct imaging systems: (a) an intravascular digital optical imaging system; (b) an external, non-invasive optical imaging system; and (c) a minimal invasive percutaneous optical imaging system. All of the three systems can be used for monitoring vascular gene therapeutic procedures and imaging of the internal elastic lamina of any small-, middle-, and large-sized vessels, including arteries and veins as well as capillaries.

In a preferred embodiment, the invention provides for a digital imaging system for intravascularly tracking vascular gene therapeutic procedures and/or imaging the internal elastic lamina (IEL) of the deep-seated vessels, including arteries and veins, such as e.g. to diagnosis or treat atherosclerosic cardiovascular disease. In one aspect, a preferred system of the invention comprises one or more of:

a. A central processing unit: e.g. a computerized control/display system;

b. A digital angioscope: e.g. an angioscope operated under the control of the central processing unit. The angioscope either transfers light (activating radiation) to excite extrinsic fluorescence or intrinsic (auto) fluorescence within and detect the fluorescent signals emitted from the target vessel wall. A preferred optical rotating probe is equipped at the tip of the digital angioscope to generate vessel wall images at a 360° view (a cross-sectional view);

c. An digital optical camera: e.g. a charge-coupled device (CCD) detector, which is connected between the central processing unit and the angioscope. The camera can be sensitive to: (i) the extrinsic/intrinsic fluorescent materials, such as the autofluorescence emitted from the fiber tissues; and (ii) an optical imaging marker, such as fluorescent proteins (green-, blue-, red-, cyan-, and yellow fluorescent proteins) and luminous materials, delivered into the vessel wall;

d. An irrigation system: e.g. an infusion pump controlled by the central processing unit. This system provides pulsatile introduction of saline or carbon dioxide (CO₂) gas to the target vessel to create a clear viewing field within the vessel for the digital angioscope; and

e. Gene/drug delivery: e.g. a mechanism or apparatus for intravascular delivery of fluorescence-marked gene/drugs or other detectably-labeled gene/drugs to the wall of the target vessel.

In another preferred embodiment, the invention provides for an external digital optical imaging system for externally tracking vascular gene therapeutic procedures and imaging of the internal elastic lamina of the surface vessels (arteries and veins as well as capillaries). The system suitably comprises one or more of:

a. A central processing unit: e.g. a computerized control/display system;

b. An external optical camera: e.g. probe operated under the control of the central processing unit, preferably a portable probe such as a hand-held probe. The probe suitably transfers external light (activating radiation) to excite the extrinsic fluorescence or autofluorescence or other detection signal within a patient and detect the fluorescent or other detection signals emitted from the vessel wall. An external optical imager can generate vessel wall images in a cross-sectional view, and is connected between the central processing unit and an external light source. The imager suitably is sensitive to: (i) extrinsic/intrinsic fluorescent materials, such as autofluorescence emitted from fiber tissues (such as the internal elastic lamina); and (ii) any optical imaging markers, such as fluorescent proteins and other luminous materials, delivered into the vessel wall; and

c. Gene/drug delivery: e.g. a mechanism for intravascularly local delivery of fluorescence-marked gene/drugs or other detectably-labeled gene/drugs to the wall of the target vessel.

In one aspect of the invention, intravascular or external detection of fluorescent markers is provided, including any colored fluorescent proteins materials expressed within the target vessel wall. In another aspect of the invention, intravascular or external imaging of the internal elastic lamina is provided, e.g. for early diagnosis of atherosclerosis.

In a preferred embodiment, the invention applies to any organs and systems of human and animal (mammal) bodies that (i) can be treated with fluorescence-gene/drug therapies or other detectable gene/drug therapies, and (ii) have autofluorescence emission from the natural tissue components of these organs and systems. The methods of the invention also further apply to detection of any extrinsic fluorescence, for example, any colored fluorescent proteins and luminous materials, delivered into the human and animal bodies.

In another preferred embodiment, the invention provides for a method for detecting and imaging of intravascularly administered molecules, said method comprising a digital optical imaging system, wherein:

a molecule with a fluorescent component, administered intravascularly to a patient, is excited by an external light source (activating radiation with a selected wavelength, thereby emitting fluorescent signals; suitably where the fluorescent signals are guided (inputted) to a charge coupled device detector; and,

the charge coupled device routes images obtained from said fluorescent signals to a central processing unit; wherein,

the central processing unit preferably magnifies, analyzes, stores and/or displays the images.

In one aspect of the invention the central processing unit is a computerized control and/or display system and has programmable software that increases magnification of an image, analyzes images, stores the images and/or displays the images on a computer monitor. A digital angioscope is also preferably in communication with the central processing unit.

In another aspect of the invention one end of the digital angioscope is coupled via a lens and filter system to the photosensitive charge-coupled device and the tip end is inserted into an intravascular location. The tip region of the digital angioscope suitably comprises an optical rotating probe to generate vessel wall images at a 360° view and the digital angioscope transfers light from a light source to the location of molecules with a fluorescent component, thereby exciting these molecules and detecting the fluorescent signals emitted from the location of these molecules.

The optical rotating probe can be either equipped at the tip of the digital angioscope, or can be used solely in any naturally existing anatomic channels, for example, such as but not limited to vessels and cavities of a mammalian body.

A probe suitably is a semi-flexible device and can be designed in any sized diameter to fit in any sized vessels, such as arteries and veins as well as capillaries. The probe can be easily combined with any existing intravascular interventional approaches, such as balloon angioplasty, stent placement, angioscopic examination, and the like. The probe can be placed into the targeted vessels under guidance by any imaging modalities. The probe suitably transmits the excitation light (activating radiation into the target to excite the fluorescent/luminescent molecules and the probe collects the fluorescent/luminescent lights emitted from the transgene-target, transfer the light signals to a CCD camera, and then routes them into the computer.

In another aspect of the invention, the tip of the probe is preferably coated with alloy markers to enable the visualization of the probe during positioning under any imaging modalities.

In another aspect of the invention, the digital angioscope transfers light from a light source to excite a molecule with a fluorescent component and detect auto fluorescent signals emitted from cells in a target vessel wall.

In a preferred embodiment the digital optical imaging system is comprised of: an external light source; a fiberoptic light guide; wavelength selective optical filters; a relay lens; and, a highly sensitive charge-coupled device detector. The external light source is a halogen lamp, xenon, infrared, or a laser generated beam. The selection of the preferable external light source depends on the optical imaging markers used, such as but not limited to fluorescence and luminescence materials.

In another preferred embodiment, the fiberoptic light guide, guides photons having wavelengths of the selected external light source and the fiberoptic light guide is flexible and dimensioned to fit into a blood vessel, which includes veins, arteries, capillaries.

In another preferred embodiment, the wavelength selective optical lenses determine the wavelength of light passing through said lenses and the preferred wavelength of light selected.

In another embodiment, the invention provides for a highly sensitive charge-coupled device detector (CCD). Preferably, a highly sensitive charge-coupled device detector such as a detector comprising arrays of at least 1024×1024 pixels.

In another aspect of the invention, digital optical imaging device is connected to a computer wherein images obtained from said digital optical imaging device are analyzed, stored and displayed.

In another preferred embodiment, the invention provides for a non-invasive method for externally detecting and imaging of gene therapeutic procedures or molecules, said method comprising a digital optical imaging system, wherein:

a molecule with a fluorescent component administered to a patient, is excited by an external light source with a selected wavelength, thereby emitting fluorescent signals; whereby,

the fluorescent signals are guided to a photosensitive charge coupled device detector via a hand held probe which is in contact with a patient's skin; and,

the photosensitive charge coupled device routes images obtained from said fluorescent signals to a central processing unit; wherein,

the central processing unit preferably magnifies, analyzes, stores and/or displays the images.

In one aspect of the invention, the external digital optical imager comprises a probe, preferably configured to be portable or hand-held, suitably operated under the control of or otherwise in communication with the central processing unit. Preferably, the probe comprises a spatially coherent fiber bundle. The hand held probe either transfers external light to excite the extrinsic fluorescence or autofluorescence within a cell or detects the fluorescent signals emitted from the vessel wall. Preferably, the hand held can detect fluorescence up to about 10 mm in depth in the skin without any interference from patient cellular or tissue components that are in the area between the location of the molecules with a fluorescent component and the external layer of the patient's skin.

In another preferred embodiment, the invention provides for a minimally invasive method for transpercutaneously tracking vascular gene therapeutic procedures and imaging of the internal elastic lamina of the surface vessels, the method comprising use of a digital optical imaging system, wherein:

a molecule with a fluorescent component administered to a patient, is excited by an external light source with a selected wavelength, thereby emitting fluorescent signals; whereby,

the fluorescent signals are guided to a photosensitive charge coupled device detector via a percutaneous fiber-optic-probe which is positioned transpercutaneously nearby the transgene-targeted vessel of the body; and,

the photosensitive charge coupled device routes images obtained from said fluorescent signals to a central processing unit; wherein,

the central processing unit preferably magnifies, analyzes, stores and/or displays said images.

The minimal invasive percutaneous optical imaging system suitably comprises a fiber-optic probe which suitably can be of any of a variety of sizes. The fiber-optic-probe can be inserted into the body via either a minimally surgical incision on the skin, or via any transpercutaneously interventional approaches under any imaging, for example, such as but not limited to CT, MRI, ultrasound, and x-ray fluoroscopy, therefore positioning the tip of the probe nearby the transgene-targeted vessel.

The distal portion of the probe is coated with alloy markers, which function as imaging markers to be visualized by different imaging modalities, thereby facilitating precise positioning of the probe toward to the target.

The fiber-optic probe suitably can not only transmit the excitation light (activating radiation) into the target, but also collect the transgene-expressed fluorescent/luminescent light and guide it to the photosensitive charge coupled device detector, and thereafter route the fluorescent/luminescent signals to a central processing unite.

In another aspect of the invention, the light source is a ring beam, a Gaussian beam or a flat beam.

In another aspect of the invention, the percutaneous fiber-optic-probe generates vessel wall images in a cross-sectional view, and is connected between the central processing unit and an external light source. Preferably, the probe is highly sensitive to extrinsic and/or intrinsic fluorescent materials.

In another aspect of the invention, the extrinsic fluorescent components include any optical imaging markers, such as but not limited to any colored fluorescence and bioluminescence (for example, green-, red-, blue-, green-, cyan-, and yellow-fluorescent markers, enhanced versions, fragments or mutations thereof), delivered into the blood vessel wall.

In a preferred embodiment, the methods of the invention are used to detect and image the location of a fluorescent molecule in any organ or body part. The methods are also used to diagnose cancer by use of fluorescently labeled antibodies, specific for cancer antigens or biomarkers. In another aspect of the invention the molecules with fluorescent components can be vectors, antibodies, drugs, biomarkers, nucleic acids, proteins, peptides, amino acids or fragments thereof.

References herein to “radiation”, “activating radiation”, “light” or the like in appropriate context refer to energy applied an administered composition to generate a detectable output from the composition, such as fluorescence. Thus, for example, activating radiation is suitably projected from a light source (visible, UV, etc.) to generate detectable fluorescence from the administered composition.

Other aspects of the invention are disclosed infra.

### BRIEF DESCRIPTION OF THE DRAWINGS

FIG. 1 is a photograph showing: (A) An optical image of an arterial tissue, 25 m in thickness, mounted on a histological glass. A strong fluorescent signal ring (arrows) outlines the intima layer. (B, 10X and C, 20X) Corresponding fluorescent microscopic findings of the same arterial tissue. A strong autofluorescent ring (B, arrows) is outlined on the intima and is primarily emitted from the internal elastic lamina (C, between arrowheads).

FIG. 2 is a schematic representation which illustrates the setup of the intravascular digital optical imaging system. Light enters the system from the light source through a fiberoptic guide. The excitation light is reflected up to the arterial lumen and the arterial wall. Light that is absorbed by the sample and reemitted as fluorescence is transmitted back through the dichroic and emission filters to the digital detector.

FIG. 3 is a schematic illustration of the hand-held or external digital optical imager. The CCD detector of the imager detects the fluorescent signal from the surface vessels, such as the carotid arterial wall, which then is routed to computer display for analysis.

FIG. 4 is a photograph showing the surgical-based gene delivery into the carotid artery (long arrow). After isolating the artery with two Sentinel loops (arrowheads), the GFP-vector solution (block arrow) was injected into the arterial lumen.

FIG. 5 is a photograph showing the external optical imaging system, which consists of: A, halogen light source (a), fiber-optic bundle (b), CCD camera (c), optical lenses (d), custom filter slider (e), fiber-optic ring-light (f). B, Filter slider removed with an emission filter seated in center slot. C, Front view of camera mounted with fiber-optic ring-light, filter slider, and optical lenses

FIG. 6 are photographs showing optical images of surgically exposed carotid arteries of a rabbit (arrowheads). A, the untransferred or control artery imaged at two-second exposure. The thin, bright lines are artifacts that resulted from fluid contacting the transparent film covering the ring-light. B, C, and D, red fluorescent protein (RFP)-transferred arteries imaged at 2, 5, and 10-second exposures, respectively. Bright areas of RFP fluorescence (block arrows) expanded as exposure time increased. No fluorescence was observed in the untransferred tissue.

FIG. 7 are photographs showing the surface plots of intensity across the width of a section of the vessel from FIG. 6(a) and (b). Intensity was flat across the control artery (a), but heterogeneous for the target artery (b) due to contribution from RFP fluorescence. The dashed line was added to denote the vessel boundary.

FIG. 8 is a photograph showing the confocal microscopy images of the results of the experiment. Panels A and B show untransferred and RFP-transferred carotid arteries, respectively. Panels C and D show untransferred and GFP-transferred femoral arteries, respectively. SMCs of transferred arteries exhibited significantly greater signal intensities due to fluorescence emitted by RFP and GFP (arrows). Background levels from autofluorescence was detected in untransferred tissue.

FIG. 9 shows photographs of images of histological sections of (a) control and (b) immunohistochemically stained arterial tissue. Stained GFP forms brown colored precipitates, resulting in a color change of the entire arterial wall from gray (a) to brown (b). 200× magnification.

FIG. 10 is a photograph showing a gelatin matrix phantom with an embedded capillary tube.

FIG. 11 is a photograph showing an external view of the optical imaging system mounted on a stand, enabling the placement of ring light on the phantom surface.

FIG. 12 is a photograph showing in panel (A): GFP signal is detected from the capillary tube located 5-mm in depth on the gelatin phantom. Panel (B): There is no signal detected from control (PBS).

FIG. 13 is a graph showing remitted fluorescence as a function of radial distance at 5 mm depth for ring light geometry. Fourth order polynomial line height delineates the beam profile.

FIG. 14 is a graph showing the fluence distribution as a function of depth at the central axis, r=0.0 cm.

FIG. 15 is a graph showing the fluence distribution as a function of depth at radial distance, r=0.8 cm.

FIG. 16 is a schematic illustration of the intravascular or minimally-invasive transpercutaneous optical imaging system for tracking vascular gene expression. Fiber optic source diameter can be any size.

FIG. 17 is a schematic illustration showing in panel (a) intravascular probe head; and panel (b) total contact area on the inner surface of the vessel.

FIG. 18 is a graph showing the fluence distribution as functions of depth at the central axis and at the periphery of the 5 mm diameter percutaneous probe (modeled in a plaque-bearing artery).

FIG. 19 is a graph showing the fluence distribution as a function of depth at the central axis of a Gaussian beam with a 1.5 mm diameter.

FIG. 20 is a graph showing the fluence distribution as a function of depth at the central axis of a Gaussian beam with a 1.5 mm diameter.

FIG. 21 is a schematic illustration showing the head of the minimal invasive transpercutanous fiber-optic-probe. The head of the probe is placed nearby the transgene-targeted vessel wall, via a surgical minute incision on the skin surface and any transpercutaneous interventinal devices, to detect fluorescent or luminescent light expressed from the transgenes.

### DETAILED DESCRIPTION OF THE INVENTION

In preferred aspects, the invention provides methods for the intravascular, external, or minimally-invasive transpercutaneous tracking of vascular gene expression and early diagnosis of disorders such as atherosclerosis using digital imaging techniques. References to a digital imaging technique generally indicate that obtained data is manipulated through a computer.

Preferred intravascular digital optical imaging systems of the invention comprise a digital angioscope preferably with a pulsatile saline or CO₂ gas infusion, a highly sensitive CCD camera, and a central processing unit to control and display fluorescent images. This system is designed for optical imaging of extrinsic/intrinsic fluorescent/luminous signals emitted from deep-seated vessels. The external digital optical imaging system consists of a hand-held probe modified from a CCD camera, and a central processing unit to control and display the optical images. This system is designed for optical imaging of extrinsic/intrinsic fluorescent/luminous signals emitted from superficially-located vessels. The minimally-invasive, transpercutaneous optical imaging system consists of a fiber-optic-probe, a CCD camera, and a central processing unit to control and display the optical images. The miminal-invasive optical imaging system can be inserted into the body via any existing transpercutaneous interventional approaches, or via any naturally-existing channels and cavities of the body. This system is designed for optical imaging of extrinsic/intrinsic fluorescent/luminous signals emitted from both deeply-seated and superficially-located vessels. Those techniques are combined with vascular gene/drug delivery procedures, in which any colored fluorescent proteins and other luminous materials are used as a marker that is therefore detected by the three digital optical imagers. In addition, these digital optical imagers can be used to detect autofluorescence emitted from the internal elastic lamina (IEL) of the target arterial wall, and thus examine early morphological changes in the IEL due to atherosclerotic involvement.

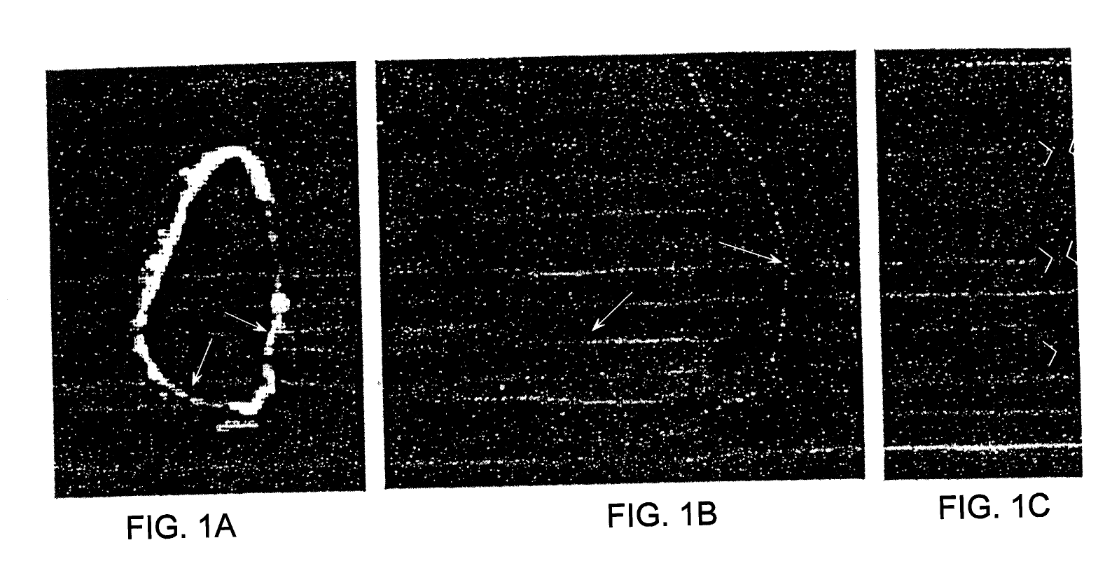

Atherosclerosis usually first involves the IEL, and any morphological changes in the IEL reflects the early development of atherosclerosis. Since the digital optical imaging system provides the specific ability to detect the morphological changes in the IEL (FIG. 1), this imaging modality can be used to diagnose atherosclerosis early and therefore promptly prevent the subsequent development of atherosclerotic diseases. The methods of the invention are therefore useful, for example, for the management of cardiovascular gene therapy to prevent restenosis after intravascular interventions, such as balloon angioplasty and stenting, as well as for the early diagnosis of atherosclerosis.

In particular, the invention includes a non-invasive method for optical imaging for determining the position of a vector used in gene therapy, a minimally invasive method for optical imaging for determining the position of a vector used in gene therapy, and an intravascular method for optical imaging for determining the position of a vector used in gene therapy.

The non-invasive method for imaging the position of a vector used in gene therapy is comprised of a few components, such as a central processing unit; a digital angioscope; an optical camera; an irrigation system and gene/drug/antibody delivery.

A brief description of the minimally invasive method used, which follows, is not meant to limit or construe the invention in any way but merely serves to illustrate a working example of the invention. Briefly, the methods of the invention for determining the position of a vector used in gene therapy are comprised of a central processing unit such as a computerized control/display system. Coupled to this system is a percutaneous fiber-optic-probe. The probe either transfers the light to excite extrinsic fluorescence or intrinsic (auto) fluorescence within and is used to detect the fluorescent signals emitted from the target vessel wall.

A brief description of the intravascular method used, which follows, is not meant to limit or construe the invention in any way but merely serves to illustrate a working example of the invention. Briefly, the methods of the invention for determining the position of a vector used in gene therapy are comprised of a central processing unit such as a computerized control/display system. Coupled to this system is a digital angioscope. The angioscope is operated under the control of the central processing unit. The angioscope either transfers the light to excite extrinsic fluorescence or intrinsic (auto) fluorescence within and is used to detect the fluorescent signals emitted from the target vessel wall. An optical rotating probe is equipped at the tip of the digital angioscope to generate vessel wall images at a 360° view (a cross-sectional view).

The optical camera suitably comprises a charge-coupled device (CCD) detector, which is suitably connected between the central processing unit and an angioscope. The camera is preferably sensitive to materials such as, for example, extrinsic and/or intrinsic fluorescent materials, such as the autofluorescence emitted from the fiber tissues; and any optical imaging markers, such as fluorescent proteins (green-, blue-, red-, cyan-, and yellow fluorescent proteins) and luminous materials, delivered into the vessel wall.

The irrigation system suitably comprises of an infusion pump controlled by or otherwise in communication with the central processing unit. This system provides pulsatile introduction of an irrigant such as saline or carbon dioxide (CO₂) gas to the target vessel to create a clear viewing field within the vessel for the digital angioscope. In order to detect the gene or drug being administered to a patient in need of such treatment, a mechanism or apparatus for intravascular delivery of fluorescence-marked gene/drugs to the wall of the target vessel is preferably employed.

Non-invasive methods of the invention suitably comprise an external digital optical imaging system for externally tracking vascular gene therapeutic procedures and imaging of the internal elastic lamina of the surface vessels (arteries and veins). This system is comprised of a central processing unit; an external optical camera; and gene/drug/antibody delivery.

The central processing unit suitably comprises a computerized control/display system and an external optical camera The external probe (preferably portable, such as a hand-held probe) is suitably operated under the control of the central processing unit. The camera suitably transfers the external light to excite the extrinsic fluorescence or autofluorescence within and detect the fluorescent signals emitted from the vessel wall. The external optical imager preferably can generate vessel wall images in a cross-sectional view, and is connected between the central processing unit and an external light source. The imager preferably is highly sensitive to, for example, extrinsic and/or intrinsic fluorescent materials, such as autofluorescence emitted from fiber tissues (for example, the internal elastic lamina); and any optical imaging markers, such as fluorescent proteins and other luminous materials, delivered into the vessel wall. The gene/drug/antibody delivery into a patient in need of such therapy incorporates a mechanism for intravascularly local delivery of fluorescence-marked gene and/or drugs to the wall of the target vessel.

Generally, the nomenclature used herein and many of the fluorescence, computer, detection, chemistry and laboratory procedures described below are those well known and commonly employed in the art. Standard techniques are usually used for chemical synthesis, fluorescence, optics, molecular biology, computer software and integration. Generally, chemical reactions, cell assays and enzymatic reactions are performed according to the manufacturer's specifications where appropriate. The techniques and procedures are generally performed according to conventional methods in the art and various general references. (Lakowicz, J. R. Topics in Fluorescence Spectroscopy, (3 volumes) New York: Plenum Press (1991), and Lakowicz, J. R. Emerging applications of fluorescence spectroscopy to cellular imaging: lifetime imaging, metal-ligand probes, multi-photon excitation and light quenching. Scanning Microsc. Suppl. Vol. 10 (1996) pages 213-24, for fluorescence techniques; Sambrook et al. Molecular Cloning: A Laboratory Manual, 2^{nd} ed. (1989) Cold Spring Harbor Laboratory Press, Cold Spring Harbor, N.Y., for molecular biology methods; Cells: A Laboratory Manual, 1^{st} edition (1998) Cold Spring Harbor Laboratory Press, Cold Spring Harbor, N.Y., for cell biology methods; Optics Guide 5 Melles Griot™ Irvine Calif., and Optical Waveguide Theory, Snyder & Love published by Chapman & Hall for general optical methods, which are incorporated herein by reference.

In another aspect of the invention a combination of the above-methods may be used to image the position of a vector used in gene therapy or a fluorescently labeled drug. The imaging methods of the invention are not limited to diseases of the blood vessel, but can be used in any gene therapy methods for treatment of diseases including tumors such as in breast cancer, liver cancer, prostate cancer, lymphomas and the like.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

The term “DNA construct” and “vector” are used herein to mean a purified or isolated polynucleotide that has been artificially designed and which comprises at least two nucleotide sequences that are not found as contiguous nucleotide sequences in their natural environment.

As used herein, the term “administering a molecule to a cell” (e.g., an expression vector, nucleic acid, a angiogenic factor, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

As used herein, “molecule” or “composition” or similar term unless otherwise indicated is used generically to encompass any vector, antibody, protein, drug and the like which are used in therapy and can be detected in a patient by the methods of the invention. For example, multiple different types of nucleic acid delivery vectors encoding different types of genes which may act together to promote a therapeutic effect, or to increase the efficacy or selectivity of gene transfer and/or gene expression in a cell. The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations, polypeptides; polysaccharides; lipopolysaccharides, viral formulations (e.g., including viruses, viral particles, artificial viral envelopes and the like), cell delivery vehicles, and the like.

The vectors are provided for example for treating diseases and the methods of the invention can detect, localize, track and image the vectors which also express fluorescent components. The vectors or molecules are comprised, for example of a gene which can prevent, correct, or normalize or improve, an abnormal condition including, but not limited to, hypertension, atherogenesis, thrombosis, intimal hyperplasia, restenosis following angioplasty or stent placement, ischemia, neoplastic diseases (e.g. tumors and tumor metastasis), benign tumors, connective tissue disorders (e.g. rheumatoid arthritis, atherosclerosis), ocular angiogenic diseases (e.g. diabetic retinopathy, macular degeneration, corneal graft rejection, neovascular glaucoma), cardiovascular disease, cerebral vascular disease, diabetes-associated disease and immune disorders. The methods of the invention determine whether the gene has been targeted to the intended tissue, cell, organ, blood vessel walls, etc., and has not become systemically diffused throughout the anatomy of the patient. This has the added benefit of determining doses and types of vectors which can be more efficacious in treating a disease by gene therapy.

As used herein, “patient” refers to any animal or mammal, especially a human.

The invention provides for intravascular, external, or minimally-invasive transpercutaneous detection of fluorescent markers, including any colored fluorescent proteins materials, expressed within the target vessel wall; and intravascular, external, or minimally-invasive transpercutaneous imaging of the internal elastic lamina to provide early diagnosis of, for example, atherosclerosis. The invention further applies to any organs and systems of human and animal bodies that can be treated with fluorescence-gene/drug therapies, and have autofluorescence emission from the natural tissue components of these organs and systems, or can be used in the early diagnosis of diseases such as cancer by using and quantitatively detecting fluorescently labeled antibodies or markers specific for a certain type of cancer such as breast, prostate, etc. Any therapeutic molecule that can be fluorescently labeled and is used for treatment of any disease can be monitored using the methods of the present invention. These inventions also further apply to detection of any extrinsic fluorescence (such as any colored fluorescent proteins) and luminous materials delivered into the human and animal bodies.

Vectors can be constructed which also comprise a detectable/selectable marker gene. In preferred embodiments these marker genes are fluorescent proteins such as green fluorescent protein (GFP), cyan-(CFP), yellow-(YFG), blue-(BFP), red-(RFP) fluorescent proteins; enhanced green fluorescent protein (EGFP), EYFP, EBFP, Nile Red, dsRed, mutated, modified, or enhanced forms thereof, and the like.

As used herein, the “green-fluorescence protein” is a gene construct which in transfected or infected cells, respectively, shines green under light and thus enables the detection of a cell transfected or infected, respectively, with GFP in a simple manner.

The term “fluorescent component” refers to a component capable of absorbing light and then re-emitting at least some fraction of that energy as light over time. The term includes discrete compounds, molecules, naturally fluorescent proteins and macro-molecular complexes or mixtures of fluorescent and non-fluorescent compounds or molecules. The term “fluorescent component” also includes components that exhibit long lived fluorescence decay such as lanthanide ions and lanthanide complexes with organic ligand sensitizes, that absorb light and then re-emit the energy over milliseconds.

As used herein, the term “FRET” refers to fluorescence resonance energy transfer. For the purposes of this invention, FRET refers to energy transfer processes that occur between two fluorescent components, a fluorescent component and a non-fluorescent component, a luminescent component and a fluorescent component and a luminescent component with a non-fluorescent component.

As used herein, the singular forms “a”, “an” and “the” include plural referents unless the context clearly dictates otherwise.

Fluorescence labeling is a particularly useful tool for marking a protein, cell, or organism of interest. Traditionally, a protein of interest is purified, then covalently conjugated to a fluorophore derivative. For in vivo studies, the protein-dye complex is then inserted into cells of interest using micropipetting or a method of reversible permeabilization. The dye attachment and insertion steps, however, make the process laborious and difficult to control. An alternative method of labeling proteins of interest is to concatenate or fuse the gene expressing the protein of interest to a gene expressing a marker, then express the fusion product. Typical markers for this method of protein labeling include -galactosidase, firefly luciferase and bacterial luciferase. These markers, however, require exogenous substrates or cofactors and are therefore of limited use for in vivo studies.

A marker that does not require an exogenous cofactor or substrate is the green fluorescent protein (GFP) of the jellyfish Aequorea victoria, a protein with an excitation maximum at 395 nm, a second excitation peak at 475 nm and an emission maximum at 510 nm. Green fluorescent protein is a 238-amino acid protein, with amino acids 65-67 involved in the formation of the chromophore.

Uses of green fluorescent protein for the study of gene expression and protein localization are well known. The compact structure makes GFP very stable under diverse and/or harsh conditions such as protease treatment, making GFP an extremely useful reporter in general.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments.

As used herein, an “antigenic determinant” is the portion of an antigen molecule that determines the specificity of the antigen-antibody reaction. An “epitope” refers to an antigenic determinant of a polypeptide. An epitope can comprise as few as 3 amino acids in a spatial conformation which is unique to the epitope. Generally an epitope consists of at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by H. Mario Geysen et al. 1984. Proc. Natl. Acad. Sci. U.S.A. 81:3998-4002; PCT Publication No. WO 84/03564; and PCT Publication No. WO 84/03506.

Antibodies directed against surface antigens for detection of tumors and the like membrane proteins can also be fluorescently labeled. Specificity for particular cell types is likely to be easier to achieve with antibodies than with other molecules because antibodies can be raised against nearly any surface marker. Also, microinjected antibodies could label sites on the cytoplasmic face of the plasma membrane, blood vessels and the like.

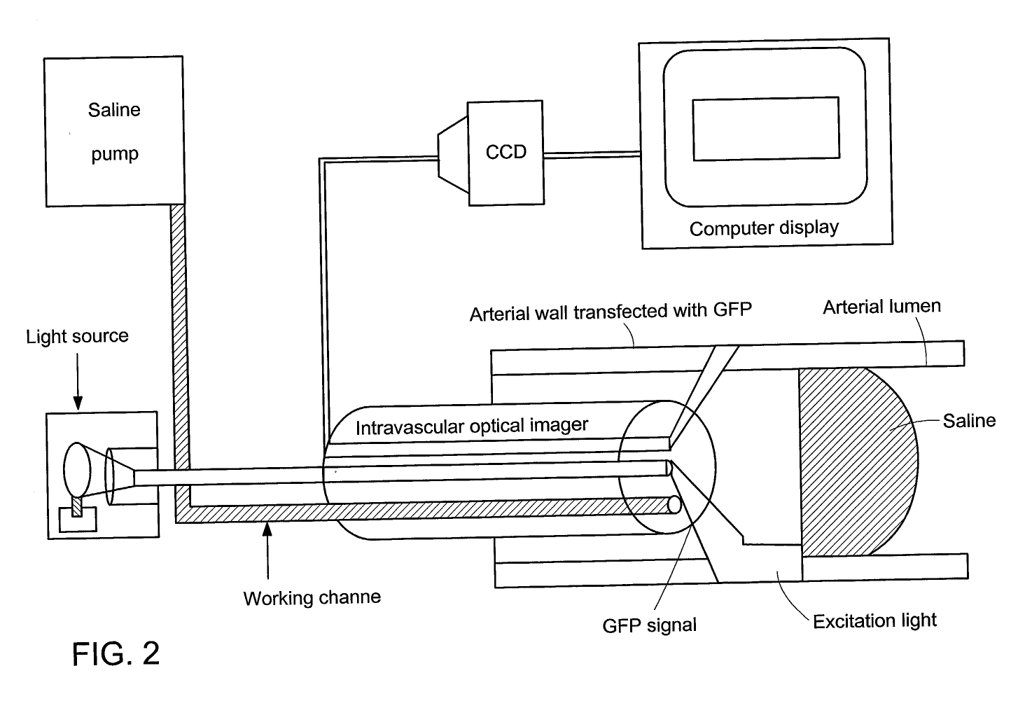

The in vitro and in vivo applications for either an intravascular, an external, or minimal invasive transpercutaneous digital optical imaging technique are many. For example, to monitor cardiovascular or vascular gene therapy. The intravascular digital optical imager (probe) can be used to detect GFP-gene expression in deep-seated vessels such as the aorta and the iliac arteries; and the external digital optical imager can be used to detect GFP-gene expression in surface vessels such as the carotid and brachial arteries. The intravascular digital optical imaging tool of the invention quantitatively detects fluorescent signals emitted from fluorescence-targeted vessel wall as shown in FIG. 2 and is therefore, a very effective method for use in detection, diagnosis and prognosis of a disease. An illustrative example of detecting fluorescence from vessel walls is as follows. Light propagation in the vasculature is simulated by Monte Carlo modeling, in which individual packets of photons are followed as they pass through multiple scattering and absorption events. One million photons are used to characterize impulse response in the aortic vessel wall. Fluence distribution at the GFP excitation wavelength of about 488 nm is simulated for the intravascular probe on normal as well as on vessel walls with plaques. The optical properties of the aorta and fibrous plaque are presented in Table 4.

The basic mechanical-optical structure of the intravascular digital optical fluorescent imaging device is an integrated design consisting of a flexible catheter jacket with an occlusion cuff at the distal end and an optical imaging bundle extendible to about 5 cm beyond the catheter jacket. A wide-angle imaging lens is mounted onto the tip of the flexible fiber optic bundle of the angioscope as shown in FIG. 2. This provides a 360° view of the vessel lumen.

The intravascular digital optical imager preferably has a soft distal tip that allows it to follow the natural curve of the vasculature. It is suitably advanced, in coordination with an imaging modularity such under x-ray fluoroscopic guidance, over about a guide wire that is placed within the working channel of the angioscope. During image acquisition, fluorescent materials are excited by an external light source with a selected wavelength. The fluorescent signals are then guided to the photosensitive CCD detector. The readout of the fluorescent signals emitted from the target vessel wall is routed to the central processing unit for intensive magnification, analysis, storage, and display.

During imaging, saline is infused through a distal irrigation port of the working channel. The flow rate and volume of the pulsatile saline infusion is controlled by the central processing unit. Although saline is a commonly used irrigation medium for angioscopic examination, carbon dioxide (CO₂) gas can clear the lumen of blood and maintain the field of visualization more effectively than saline.

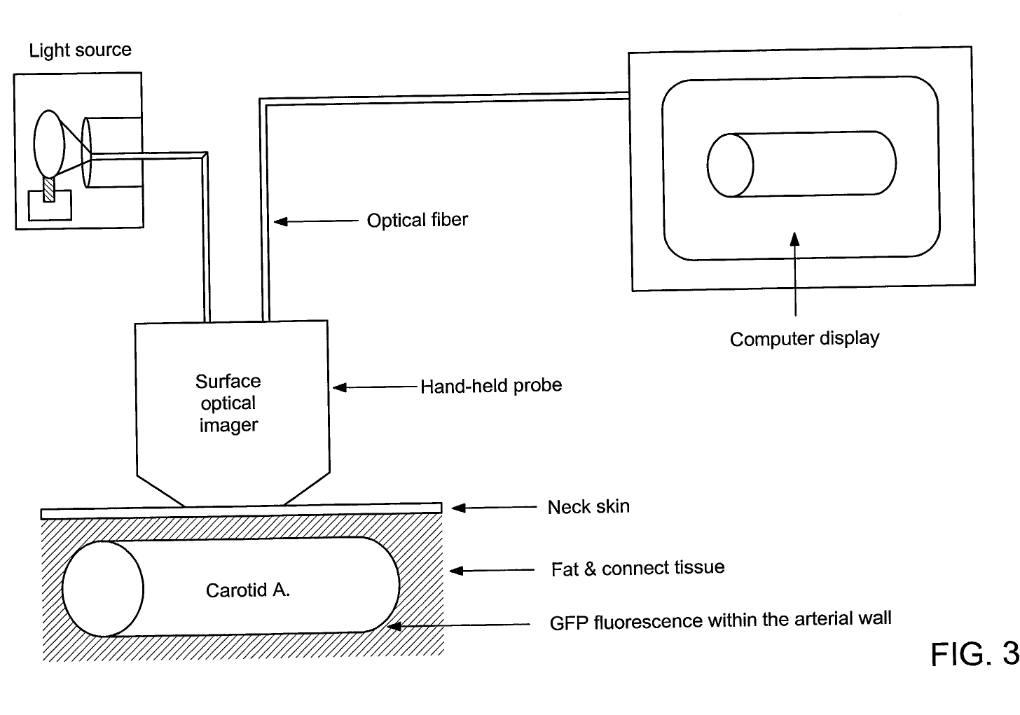

The external digital optical fluorescent imager, or non-invasive method, is a digital optical imaging system comprised of a detector module and a hand-held probe, as shown in FIG. 3. The detector module is built based on the most advanced CCD technology. The readout electronics is custom designed to minimize readout noise and a compact thermal electric cooler is employed to reduce thermal noise.

A probe which is preferably portable particularly hand-held (see FIG. 3), discussed below, is preferably a spatially coherent fiber bundle. During image acquisition, the probe is suitably placed in close contact with the neck skin area under which the target carotid artery is located. There are several layers from the skin to the carotid arterial wall, composed primarily of fat and connective tissues. The average thickness of the carotid arterial wall is about 0.7 mm. When tightly compressed with the tip of the optical probe, the distance between the targeted carotid arterial wall and the detector is, for example, about 5 to 7 mm in thickness for a 70 kg adult. The probe collects the fluorescent signals and guides the signal to the detector module for image acquisition, analysis, and display. A mathematical method is used to compensate for such signal loss during the fluorescent light transit from the target vessel wall to the detector.

Medical optical imaging can be employed to detect light signal transmitting/reflecting or emitting from scattering media, such as tissues, to determine interior structure and chemical content. Prominent advantages of the optical imaging technique of the invention include: 1) portability for bedside or operating room monitoring; 2) lack of exposure to ionizing radiation; 3) relatively low cost; and 4) the ability to directly detect signals emitted from fluorescent materials such as GFP's.

The invention also includes methods and instruments for the measurement of optical properties of tissues taken across a skin boundary, while accounting for the effects of skin layers on the properties measured. The measurement of optical properties of tissue across a skin boundary is adversely affected by the non-homogeneity of the different layers of the skin. Prior methods have ignored the effect of multiple layers of skin tissue on the measured optical properties.

To date, in vivo molecular imaging of vascular gene therapy has not been explored because of the lack of appropriate imaging modalities to address this promising new avenue of therapeutic management. The results which follow, show that the digital optical imaging system of the invention is highly sensitive to GFPs.

In one embodiment of the invention, it is preferable to be able to detect various wavelengths of the fluorescent signals, thereby eliminating any autofluorescence from tissues. In another aspect, any fluorescent material can be used, such as, for example, blue, cyan, and yellow fluorescent proteins. These fluorescent variants emit fluorescence with a wavelength different from those of current GFPs, which may benefit the differentiation of extrinsic fluorescent proteins (transfected into the target tissues) from those intrinsic autofluorescences (emitted by the tissue structures themselves).

New versions of green fluorescent protein have been developed, such as a “humanized” GFP DNA, the protein product of which has increased synthesis in mammalian cells. One such humanized protein is “enhanced green fluorescent protein” (EGFP). Other mutations to green fluorescent protein have resulted in blue-, cyan- and yellow-green light emitting versions.

Endogenously fluorescent proteins have been isolated and cloned from a number of marine species including the sea pansies Renilla reniformris, R. kollikeri and R. mullerei and from the sea pens Ptilosarcus, Stylatula and Acanthoptilum, as well as from the Pacific Northwest jellyfish, Aequorea victoria; Szent-Gyorgyi et al. (SPIE conference 1999), D. C. Prasher et al., Gene, 111:229-233 (1992) and several species of coral (Matz et al. Nature Biotechnology, 17 969-973 (1999). These proteins are capable of forming a highly fluorescent, intrinsic chromophore through the cyclization and oxidation of internal amino acids within the protein that can be spectrally resolved from weakly fluorescent amino acids such as tryptophan and tyrosine.

A variety of mutants of the GFP from Aequorea victoria have been created that have distinct spectral properties, improved brightness and enhanced expression and folding in mammalian cells compared to the native GFP, (Green Fluorescent Proteins, Chapter 2, pages 19 to 47, edited Sullivan and Kay, Academic Press, U.S. Pat. No. 5,625,048 to Tsien et al., issued Apr. 29, 1997; U.S. Pat. No. 5,777,079 to Tsien et al., issued Jul. 7, 1998; and U.S. Pat. No. 5,804,387 to Cormack et al., issued Sep. 8, 1998). In many cases these functional engineered fluorescent proteins have superior spectral properties to wild-type Aequorea GFP and are also preferred for use as reagents in the present invention.

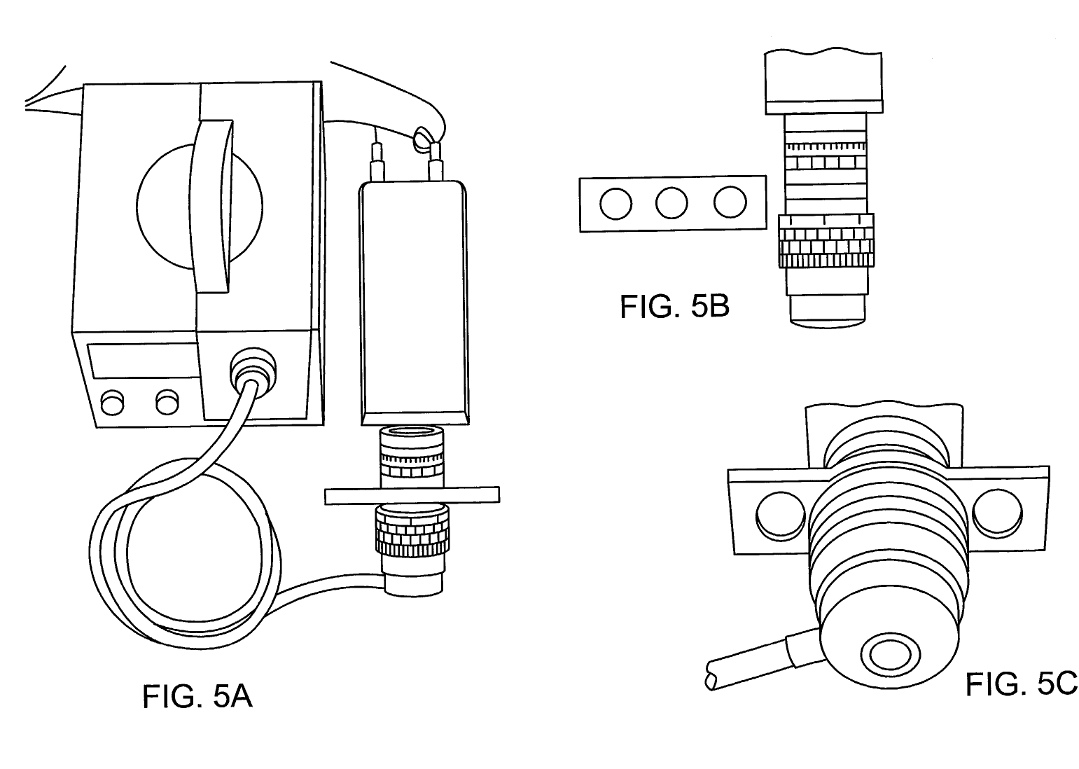

The set up of the external optical imaging system of the invention is illustrated in FIG. 5. The system preferably includes a charge coupled device (CCD) video camera (SensiCam, Cooke Corp., MI) that interfaces with an image-grabber board installed on a personal PC (Dimension 4100, Dell Computer Corp., TX). Imaging software includes, for example, IPLab Scientific Image Processing v3.070 (Scanalytics Inc., VA) and NIH Image v1.62 (U.S. National Institutes of Health, http://rsb.info.nih.gov/nih-image). A series of lenses (Fujinon-TV, Fuji Photo Optical, Japan; Nikon AF Nikkor, Nikon, Japan) and emission bandpass filters (XF3080, Omega Optical Inc., VT; HQ610/75, Chroma Technology Corp., VT) are mounted onto the camera. The emission filters are preferably selected to capture the specific fluorescence wavelengths of GFP or RFP. However, as discussed above, any wavelengths can be selected based on the fluorescent component used. Broadband light from a 250 W halogen light source (Schott KL2500 LCD, Schott Group, Germany) is filtered by excitation bandpass filters (XF1072, Omega Optical Inc., VT; HQ545/30, Chroma Technology Corp., VT) that selectively pass excitation wavelengths for GFP, RFP or any other fluorescent component. A fiber-optic ring-light (TransLite, TX) transmits filtered excitation light onto the skin or the target vessel.

An illustrative example for use of the device is detection of GFP or RFP. After peak expression of GFP or RFP, the transferred arteries can be surgically exposed and isolated for optical imaging. The camera is preferably placed directly onto the target vessel, and images are captured for various exposure times (1, 2, and 5 seconds). To visualize, for example, GFP or RFP, their respective filter sets (excitation and emission filters) are used. These filter sets can be changed to detect different excitation and emission wavelengths.

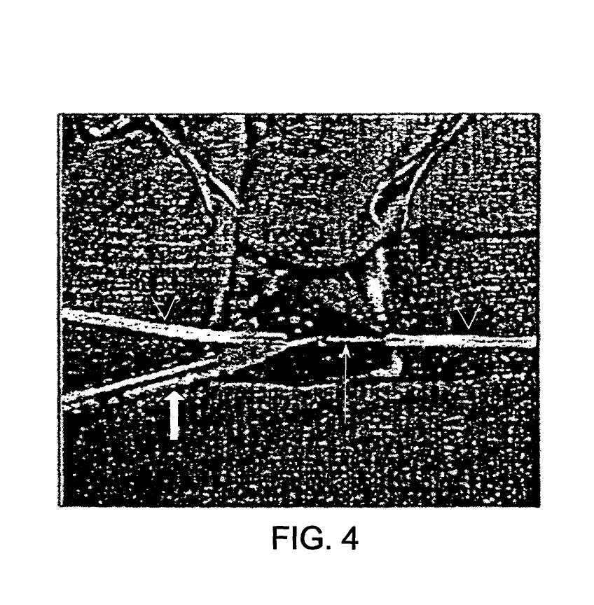

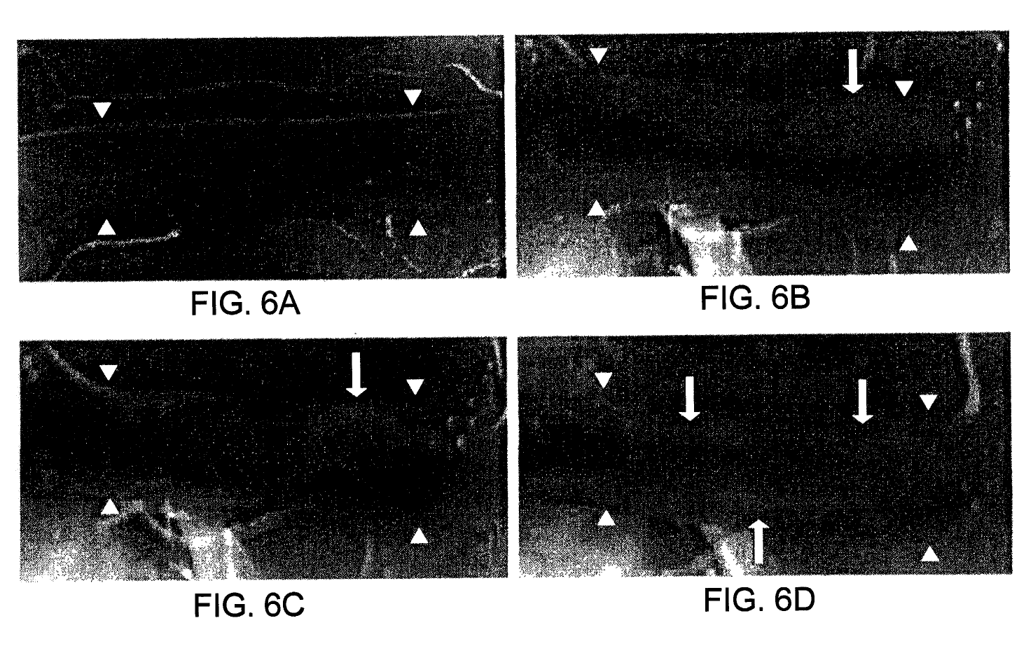

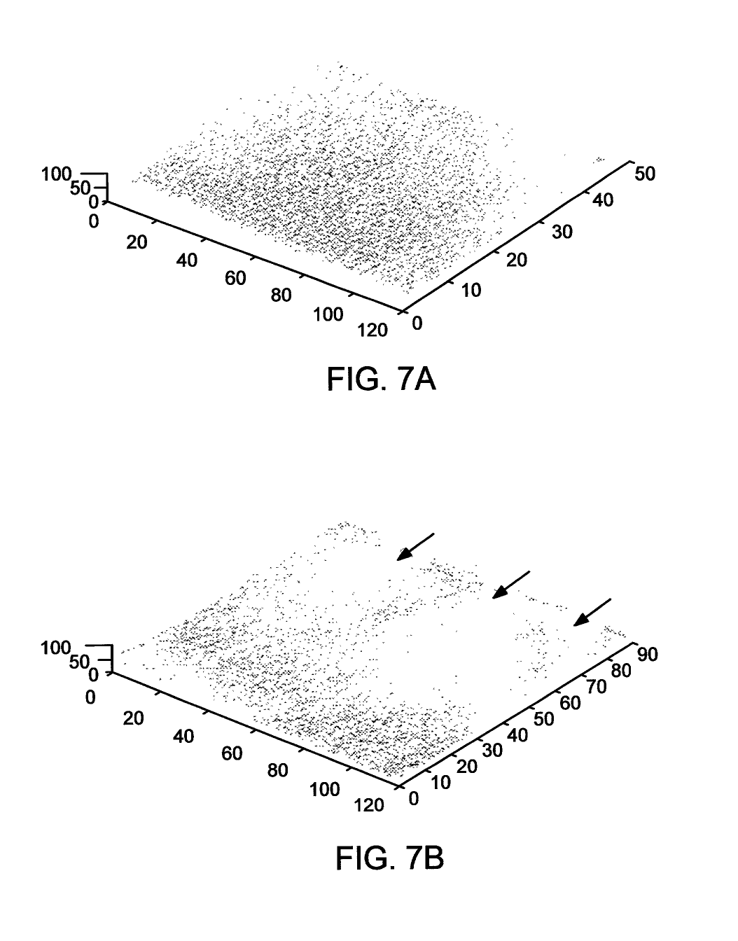

FIG. 5 is illustrative of the non-invasive optical imaging system, which is comprised of a light source (a), CCD camera (b), lenses and custom filter slider (c), fiber-optic bundle (d), and fiber-optic ring light (e). B and C show the front view of the ring-light and the filter slider with an emission filter seated in the center slot, respectively. FIG. 4, is illustrative of surgical-based gene delivery into the carotid artery (block arrow) that is isolated with two white sentinel loops and injected with an RFP-vector solution (*). FIG. 6, A-D, is illustrative of the results obtained of optical images of surgically exposed carotid arteries (arrowheads). A, The untransferred artery imaged at two-second exposure. The thin, bright lines are artifacts that resulted from fluid contacting the transparent film covering the ring-light. B, C and D, RFP-transferred arteries imaged at 2-, 5-, and 10-second exposures, respectively. Bright areas of RFP fluorescence (block arrows) expanded with longer exposure times. No fluorescence was observed in the untransferred tissue. FIG. 7, A and B, shows plots of signal intensity corresponding to a region of interest spanning the artery from images FIG. 6 A and B, above. The arrows indicate regions of high signal intensity due to RFP fluorescence.

As used herein, the expression “optical properties” refers to the absorption, scattering, emission, and depolarization properties of the tissues. The expression “optical parameter” refers to a parameter that describes and defines an optical property of a medium and its components. Examples of optical parameters include absorption coefficients, scattering coefficients, anisotropy factors, transport optical mean free path, extinction coefficients of analytes and the like. The expression “scattering medium” refers to a medium that both scatters light and absorbs light. The expression “absorption coefficient” (i.e., μₐ) refers to the probability of light absorption per unit path length. The expression “scattering coefficient ” (i.e., μₛ) refers to the probability of light scattering per unit path length.

Photon-medium interactions include (1) a scattering event followed by a scattering event and (2) a scattering event followed by an absorption event. Penetration depth is related to the change of light intensity in a scattering medium as a function of distance traveled by the light along the same path as the incident light. The expression “diffuse reflectance” means a measure of the intensity of light that is re-emitted from the surface of a sample in all directions except the direct reflection direction when the surface is illuminated by incident light. The expression “spatially resolved diffuse reflectance” refers to a measurement of light that is re-emitted from a sample and collected at several light collection sites and at a defined distance from a light introduction site. Alternatively, this expression can refer to the light collected at a given light collection site on the sample boundary as a result of introducing light at discrete light introduction sites located on the same boundary at defined distances from the light collection site. The expression “frequency domain measurement” refers to a measurement of light involving the phase angle and/or the amplitude change of modulated incident light, at a given separation distance of a light introduction site from a light collection site, as the light transverses a scattering medium. The expression “beam of light” means a group of photons traveling together in nearly parallel trajectories toward a sample and striking the surface of the sample in a predefined area only. As a practical matter, the predefined area on the surface of a sample struck by a given beam of light is that area that is covered by an illuminating element, such as an optical fiber. The expression “light introduction site” means a location on the surface of a sample, e.g., blood vessel, organs, tissue, or the like, at which light is injected or inserted into the sample. The source of the light can be located at the light introduction site or can be located remote from the light introduction site. If the source of light is located remote from the light introduction site, the light must be transmitted to the light introduction site by light transmitting means, such as, for example, optical fibers. The expression “illuminating element” means a component located at the light introduction site that delivers light to the sample, e.g., blood vessels, organs, tissue, or the like. The illuminating element is typically an optical fiber that transmits light from a source of light to the light introduction site. However, if the source of light can be located at the light introduction site, the source of light can be the illuminating element. The expression “light collection site” means a location on the surface of a sample, e.g., blood vessels, organs, tissue, or the like, at which light is that is re-emitted from the sample is accumulated. The detector, which determines the intensity of the re-emitted light, can be located at the light collection site or can be located remote from the light collection site. If the detector is located remote from the light collection site, the light must be transmitted to the detector by light transmitting means, such as, for example, optical fibers. The expression “light collecting element” means a component covering an area at the light collection site that accumulates light that is re-emitted from the sample, e.g., blood vessels, organs, tissue, or the like. The light collecting element is typically an optical fiber that transmits light from the light collection site to a detector. However, if the detector can be located at the light collection site, the detector can be the light collecting element. The term “sample” means a biological or non-biological material that scatters and absorbs light. Samples include, but are not limited to, tissue, blood, urine, and other biological solids and fluids. Samples can be homogeneous or heterogeneous and can consist of a single layer or a plurality of layers. As used herein, the term “tissue” includes tissue of any animal, including humans. Moreover, the term “tissue” is intended to include the intact tissue of a living animal, including humans. The term “distance” means (1) the distance as measured from the center of one site to the center of the other site when referring to the distance between two sites; (2) the distance from the center of one element to the center of the other element when referring to the distance between two elements; (3) the distance between the center of a given site and the center of an element not in that site when referring to the distance between a given site and an element not in that site. The expression “re-emitted light” means a group of photons emerging from a sample as a result of the scattering, reflection, absorption, and emission of the light that illuminates the sample. As used herein, the term “light” means electromagnetic radiation. Preferably, the light has a wavelength ranging from about 400 nm to about 10,000 nm, more preferably from about 400 nm to about 2500 mn, most preferably from about 500 to about 700 mn.

Absorption and scattering coefficients can be determined from the output of the apparatus of this invention, Monte Carlo modeling, and a calibration procedure. Calibration can be carried out by determining the spatially resolved diffuse reflectance values of a set of materials of known optical properties. These materials are known as tissue-simulating phantoms. They include lipid suspensions, such as Intralipid™ (Pharmacia, Clayton, N.C.) and Liposyn™ (Abbott Laboratories, North Chicago, Ill.). The lipid suspension is diluted to generate suspensions having known values of scattering coefficients. A colored compound, hemoglobin, or blood is added to the suspension to generate different values of absorption coefficients. Alternatively, plastic rods or sheets containing colored pigments can be used. Also, polished pieces of scattering glass, such as opal glass, can be used to generate reference values for absorption and scattering coefficients. Absorption coefficients and scattering coefficients of these phantoms are usually determined by independent standard optical methods.

In a preferred embodiment the methods of the invention optically image gene expression from near-surface vessels noninvasively and in vivo. Monte Carlo simulations of the phantom experiments, are used to validate and improve the performance of the optical imaging system. As used herein, the term “Monte Carlo model” is used to simulate the propagation of light transport in tissue based on tissue optical properties (e.g. absorption, scattering, and anisotropy) Monte Carlo simulations have been used extensively to assess the effects of tissue optical properties, excitation beam profiles, and collection geometries on fluorescence and is a skill well known in the art. Results from these simulations are presented in the Examples which follow. A Monte Carlo model allows, for example, calculation of the number of photons emitted from GFP or RFP and determination of what fraction is transmitted through the tissue and detected by the camera.

In preferred embodiments, the light sources used can be any light source which provide a desired excitation wavelength and radiant intensity, such as for example, a halogen lamp, Xenon lamp, infrared light, or lasers and the like, which have greater radiant intensities at the desired excitation wavelengths. The determination of the light source used will depend on the fluorescent protein used, whether the non-invasive method, or the minimally invasive method, or alternatively the intravascular method of detecting fluorescently labeled molecules are used.

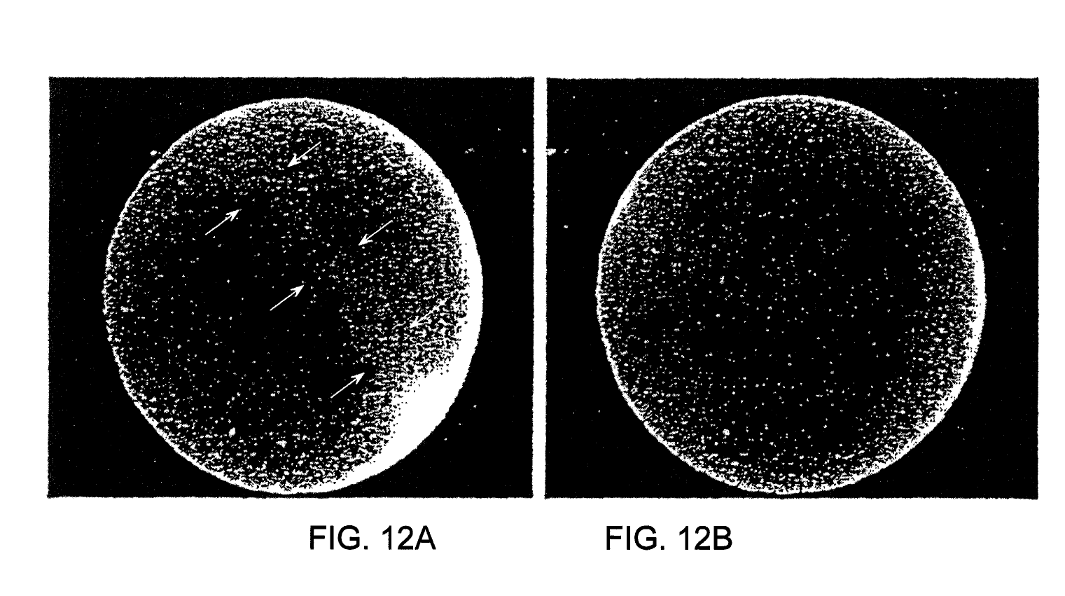

As an illustrative example, a set of gelatin matrix phantoms simulating human tissue-like properties were developed. In the phantoms, polystyrene microspheres are used as scatterers and human hemoglobin as absorbers. The human hemoglobin is purchased from Sigma Chemical Co. (St. Louis, Mo.) and is used at about 2% by volume red blood cells (RBCs) in concentration. Polystyrene microspheres at 1.05-μm diameter can be purchased from Polysciences Inc. (Warrington, Pa.), and are concentrated at about 0.625%-by volume in the sample solution. The phantom matrix is constructed with, for example, Knox unflavored gelatin in PBS solution. The fluorophores FAD and Rodamine B were not used in the sample due to possible interference with GFP fluorescence. A capillary tube was selected to simulate the arterial geometry containing the transfected fluorescent proteins in vascular gene transfection. As illustrated in FIG. 12, the capillary tube made from glass with an inner diameter of about 1.20 mm and a wall thickness of about 0.40 mm, is embedded inside the petri dishes filled with gelatin matrix at about 1, 2, 3, 5, and 7 mm depths. The tubes are first filled with PBS solution (as a control), and then are imaged using the optical imaging system of the invention. For comparison, GFP-transfected smooth muscle cells (SMC) are injected in suspension at about 8 million cells/mL into the same tube to replace the PBS. Then, optical imaging is performed to detect GFP emission from SMCs. Two concentrated GFP/SMC solutions at about 30% and 15% of transfected cells/volume can be injected to correlate with the detected fluorescent signal intensity.

As used herein, the term “Monte Carlo simulations” refers to simulations of physical processes based on a stochastic model. For example, the propagation of excitation light at some wavelength, λₘ, is first simulated into the tissue model by a Monte Carlo program and then the remitted photon propagation is modeled to the top surface of the medium at the emission wavelength, λₘ where λₘ≧λ_{X}, with a separate Monte Carlo program. That is, the Monte Carlo simulation refers to a statistical method that can be used to trace photon propagation in a scattering medium by means of numerical simulation.

A program called MCML is written in standard C language for Monte Carlo simulation of photon propagation in multi-layered tissues, which deals with only responses to an infinitely narrow photon beam normally incident on the surface of the tissue. However, all light source beam profiles have finite size beam geometry. Through a convolution process over the impulse response given by the output of MCML, the response to finite size beam geometry is computed. A typical program for implementation of the convolution process response to Gaussian beams and circularly flat beams is for example, CONV. True Gaussian beams have no “edges”; that is, the intensity of a perfect Gaussian never actually falls to zero at large distances from the center. This arises from the nature of the (circularly symmetric) Gaussian intensity profile.

The imaging device used, herein, preferably has a ring light source, which has neither a Gaussian nor a circularly flat beam profile. By utilizing the linearity and spatial invariance properties of convolution, the ring light geometry is modeled by subtracting the convolution response of a narrower beam from the response of a wider beam. By comparing the radial fluence distribution profile and the intensity profile of actual ring light illumination, the wider beam is selected to be a Gaussian beam with the outer diameter of preferably about 1.70 cm of the ring light. The narrow beam is selected to be a flat beam of about 1.0 cm diameter, which is smaller than the inner diameter, preferably about 1.56 cm of the ring light source. The presence of an edge effect due to radial diffusion in the circularly flat geometry is taken into consideration in selecting the width of the narrow flat beam. The excitation light irradiance rate is measured by a power meter and is used in the convolution process. After modeling the ring light beam response, illumination efficiencies of a ring beam, a Gaussian beam, and a circularly flat beam are compared by simulating the fluence rate as a function of depth.

Various stent types and stent constructions may be employed in the methods and systems of the invention. A stent is a generally longitudinal tubular device which is useful to open and support various lumens in the body. For example, stents may be used in the vascular system, urogenital tract and bile duct, as well as in a variety of other applications in the body. Endovascular stents have become widely used for the treatment of stenosis, strictures, and aneurysms in various blood vessels. These devices are implanted within the vessel to open and/or reinforce collapsing or partially occluded sections of the vessel. Suitable stents are generally open ended and are radially expandable between a generally unexpanded insertion diameter and an expanded implantation diameter which is greater than the unexpanded insertion diameter. Suitable stents are often flexible in configuration, which allows them to be inserted through and conform to tortuous pathways in the blood vessel. A stent is suitably inserted in a radially compressed state and expanded either through a self-expanding mechanism, or through the use of balloon catheters.

Among the various stents useful in the systems and methods of the invention include, without limitation, self-expanding stents and balloon expandable extents. The stents may be capable of radially contracting, as well, and in this sense can best be described as radially distensible or deformable. Self-expanding stents include those that have a spring-like action which causes the stent to radially expand, or stents which expand due to the memory properties of the stent material for a particular configuration at a certain temperature. Nitinol is one material which has the ability to perform well while both in spring-like mode, as well as in a memory mode based on temperature. Other materials are of course contemplated, such as stainless steel, platinum, gold, titanium and other biocompatible metals, as well as polymeric stents.

The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like.

All documents mentioned herein are incorporated herein by reference.

The following non limiting are illustrative of the invention. In the below examples, the following materials and methods were used.

### Materials and Methods

### Fluorophore Selection

Two exogenous fluorophores, GFP and red fluorescent protein (RFP), were used to compare their effectiveness as markers for gene expression. The main advantages of GFP as an imaging marker include greater GFP gene-transfection efficiency and higher quantum yield, but its emission spectra intersects the spectra of endogenous fluorophores such as collagen, which leads to greater autofluorescence or background. In contrast, RFP excitation and emission peaks have a longer wavelengths, which result in less autofluorescence and deeper penetration through tissue.

### Animals

The bilateral carotid or femoral arteries of three New Zealand white rabbits and two domestic pigs, were used. All animals were treated according to the “Principles of Laboratory Animal Care” of the National Society for Medical Research and the “Guide for the Care and Use of Laboratory Animals” (NIH Publication No. 80-23, revised 1985). The Animal Care and Use Committee of the Johns Hopkins University has approved the experimental protocol.

Animals were sedated with an intramuscular injection of a mixture of ketamine (22 mg/kg body wt), acepromazine (1.1 mg/kg body wt), and atropine (0.05 mg/kg body wt). An ear vein was cannulated, which permitted maintenance of anesthesia. Intravenous pentobarbital (20 mg/kg body wt) was administered later to bring the animal to a surgical plane of anesthesia. Animals were intubated and mechanically ventilated with 30% oxygen and 70% room air (Mark 7A Bird respirator). The animals were also heparinized (100 IU/kg). Anesthesia was monitored for the duration of the experiment using regular tests of the eyelid reflex and mild paw compression.

### Surgery-based GFP-vector Delivery

Using an arteriotomy approach, the bilateral carotid arteries, approximately 1.5-cm in length, of three rabbits were first exposed (FIG. 4). The exposed arterial portion at the right side was isolated with sutures and then harvested (prior to the administration of any GFP-vector in the left side) to serve as control. The exposed arterial portion at the left side was isolated by temporarily tightening the target vessel proximally and distally with two Sentinel loops (Sherwood Medical, St Louis, Mo.) (FIG. 24). By inserting a 24-gauge angiocath (Baxter Healthcare Corp., Marion, N.C.) into the isolated segment of the left carotid artery, the blood from the vessel lumen was drawn off, and thereafter directly injected a modified GFP-lentivirus vector solution to fill the isolated vessel portion for one hour. A silk tie was placed in the adjacent tissue to identify the vessel segment to be harvested. After the transfection, GFP-vector solution was drawn off, removed the angiocath, closed the puncture point with superglue (Elmer's Products Inc., Columbus, Ohio), untightened the Sentinel loops, closed the arteriotomy incision with sutures, and kept the rabbit alive for four days. After the surgery-based gene delivery, a postoperative analgesic (buprenorphine, 0.01 mg/kg, i.m., q12 h) was administered. At day five after GFP-vector transfection, the rabbit was anesthetized, and the GFP-targeted segment of the left carotid artery was harvested. Then, the animals were euthanized with a dose of 100 mg/kg of pentobarbital. Each of the harvested arterial specimens was cut into two equal pieces: one for optical imaging, and another for immediate frozen sections and subsequent fluorescent microscopic examination as well as immunohistochemistry confirmation.

### Catheter-based GFP-vector Delivery

To minimize the invasiveness of the gene transfer, we used a catheter-based approach to locally deliver GFP-lentiviral vectors to the left femoral arteries of two domestic pigs (20 kg in weight). A 9F introducer was cannulated into the aorta through the right carotid artery. To obtain an angiography of the pelvic and femoral arteries, a 4F pigtail angiography catheter was then positioned into the abdominal aorta, and 20 mL of 60% diatrizoate meglumine (Hypaque, Nycomed Inc., Princeton, N.J.) was injected at a flow rate of 10 mL/second. An appropriate segment (2 cm in length and 3.0-3.5 mm in diameter) of the left femoral artery was selected for gene transfer using the resulting angiogram. A balloon dilatation infusion gene delivery catheter (Remedy, Boston Scientific, Boston, Mass.) was placed at the target segment where the GFP-lentiviral vectors were delivered for 8-10 minutes. The diameter of the inflated balloon ranged from 3.5 to 4.0 mm, and the ratios, diameter of the target artery:diameter of the inflated balloon, were 3.0 mm:3.5 mm and 3.5 mm:4.0 mm. Choosing a balloon with a greater than the artery ensured proper infusion of the gene-vectors into the arterial wall. The right femoral artery was not transferred and served as the control. Optical imaging was performed on the corresponding segment of the right femoral artery and harvested prior to gene transfer.

### Optical Imaging

Optical imaging of the control or untransferred arteries was performed prior to any gene transfer. The segment of the untransferred artery corresponding to the target artery was surgically exposed and isolated for in vivo imaging. The camera, equipped with either excitation and emission filters for GFP or RFP depending on which fluorophore gene was to be transferred, was placed directly onto the vessel, and a series of images was captured with exposure times ranging from 1 to 10 seconds. When imaging of the control was completed, the control vessel was harvested, followed by gene delivery to the target artery.

On the day of peak GFP or RFP expression, the transferred arteries were imaged in the same fashion as the control. The camera was positioned to capture images of the specific area of the target vessel where gene delivery occurred. Upon completing the imaging of the transferred arteries, the animals were immediately euthanized, and the target arteries were harvested for inspection with confocal microscopy and immunohistochemistry.

### EXAMPLE 1

### A Non-Invasive Optical Imaging Method For Tracking Vascular Gene Expression In Vivo

A. Gene-Vector Transfer

Two types of fluorescent protein genes, GFP and RFP genes, were used for comparison. GFP has a greater gene transfer efficiency due to its viral vector, but its emission spectra intersect many endogenous fluorophores, resulting in more background noise or auto fluorescence. RFP excitation and emission maxima have longer wavelengths, and it has been shown that photons of longer wavelengths penetrate more deeply into tissue. The method by which the animal was prepared and the GFP vector delivered is shown in FIG. 4. Using a third-generation lentivirus, a GFP-lentiviral vector was produced (30-50×10⁶ transducing unites/mL) that was transferred into the right femoral arteries or corotid arteries of one New Zealand white rabbit and two domestic pigs. For RFP-plasmid transfection (20 μg/mL, pDsRed-N 1; Clontech Laboratories, Inc., CA), the right carotid arteries of two New Zealand white rabbits were transferred for two hours using the same surgical approach. The left femoral and carotid arteries were not transferred with any vectors or genes and served as controls. The animals were subsequently allowed to survive for 5 days to enable peak GFP expression and for 1.5 days for peak RFP expression.

B. In Vivo Optical Imaging

The optical imaging system is depicted in FIG. 5. The system included a charge coupled device (CCD) video camera (SensiCam, Cooke Corp., MI) that interfaced to an image-grabber board installed on a personal PC (Dimension 4100, Dell Computer Corp., TX). Imaging software included IPLab Scientific Image Processing v3.070 (Scanalytics Inc., VA) and NIH Image v1.62. A series of lenses (Fujinon-TV, Fuji Photo Optical, Japan; Nikon AF Nikkor, Nikon, Japan) and emission bandpass filters (XF3080, Omega Optical Inc., VT; HQ610/75, Chroma Technology Corp., VT) were mounted onto the camera. The emission filters were selected to capture the specific fluorescence wavelengths of GFP or RFP. Broadband light from a 250 W halogen light source (Schott KL2500 LCD, Schott Group, Germany) was filtered by excitation bandpass filters (XF1012, Omega Optical Inc., VT; HQ545/30, Chroma Technology Corp., VT) that selectively passed excitation wavelengths for GFP or RFP. A fiber-optic ring-light (Nevoscope, TransLite, TX) transmitted filtered excitation light onto the skin or the target vessel.

After peak expression of GFP or RFP, the transferred arteries were surgically exposed and isolated for optical imaging. The camera was placed directly onto the target vessel, and images were captured for various exposure times (1-10seconds). To visualize GFP or RFP, their respective filter sets (excitation and emission filters) were used. Upon completion of optical imaging, we euthanized the animals and harvested the control and target arteries. Both arterial specimens were cut into two equal-sized pieces: one for confocal microscopy and the other for immunohistochemical staining.

C. Confocal Microscopy

To assess the reliability of the technical development, results from optical imaging and confocal microscopy were compared. A confocal microscope (Zeiss LSM 410; Zeiss Group, Germany) was used to image and measure average intensity from sections of fresh control and fresh target arterial tissue mounted onto slides. Excitation of GFP and RFP was achieved with an Argon laser (488 nm wavelength) and a green HeNe laser (543 nm), respectively. Filters were set to maximally capture the specific emission fluorescence spectra of GFP or RFP. Individual smooth muscle cells were visualized with a 40X lens (Zeiss C-Apo X40; Zeiss Group, Germany). For each of the control and target tissues, average intensity measurements were recorded for at least 27 randomly 5 selected sites, with each site covering an area of 0.0256 mm². This process was repeated by several investigators to reduce individual subjective bias.

D. Immunohistochemistry

To confirm the success of the primary GFP/RFP-vector transfer, immunohistochemical staining using specific monoclonal antibodies for GFP (Roche, Ind.) or RFP (Clontech Laboratories, Inc., CA), was performed. Verification by immunohistochemical analysis followed previously developed methods.

For immunohistochemical detection of GFP in the tissue sections, the slides were fixed in 100% ethanol for 20 min. After air-drying, the slides were treated with 3% H₂O₂ in phosphate buffered saline (PBS, pH 7.4) for 20 minutes to quench endogenous peroxidase activity. Following blocking of non-specific sites with 10% goat serum in PBS for 60 minutes, the slides were washed with PBS (three times for five minutes) and incubated with specific monoclonal antibody for GFP (dilution 1:250; Roche, Indianapolis, Ind.) at 4° C. overnight. After washing off unbound primary antibodies with PBS, the slides were incubated with biotinylated anti-mouse antibody (1:500 dilution) for one hour. Specific binding was detected using an avidin-biotin-HRP complex (1:100 dilution) for one hour (Vector, Burlingame, Calif.), and a substrate solution of H₂O₂ and diaminobenzidine (DAB) kit according to the manufacturer's instructions. Then the slides were counterstained with hematoxylin, dehydrated through gradient alcohol and xylenes, mounted with coverslips, examined under an Olympus VANOX AHBS3 microscope. Negative controls were carried out with normal mouse IgG according to the primary antibodies used for the staining.

Results

Images of target arteries obtained by the optical imaging system showed distinct areas of fluorescence emitted from RFP and GFP (FIG. 6). In RFP-transferred arteries, the areas of RFP fluorescence were located along the arterial wall and the signal intensities within these areas were greater than the surrounding arterial tissue (FIG. 6(b-d)). The untransferred arteries showed no such areas of enhancement since no exogenous fluorophores were present (FIG. 6(a)). Comparing the images from 2-, 5-, and 10-second exposures, the areas of RFP emission expanded as the exposure time increased (FIG. 6(b-d)). It was expected that fluorescence increased with exposure time since more fluorophores were excited, leading to greater fluorescence emission and detection by the camera. FIG. 7 illustrates surface plots of intensity for a region of interest on the control and RFP-transferred arteries from FIG. 6(a)(b). The plot in FIG. 7(b) demonstrated peaks of intensity values across the width of the target vessel whereas the plot for the control in FIG. 7(a) was flat. These dramatic differences confirmed the contribution of RFP fluorescence to overall intensity.

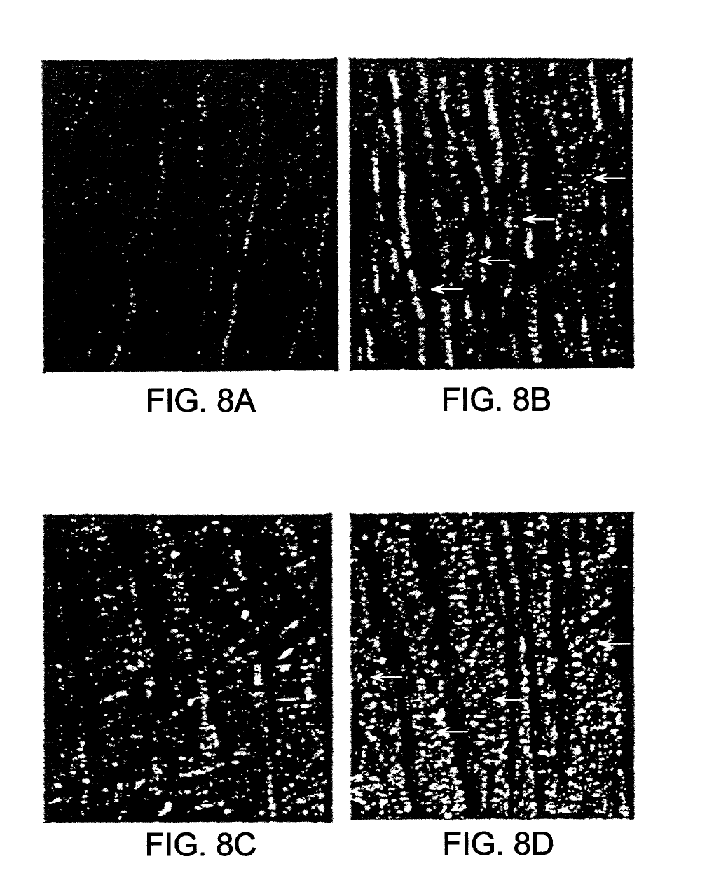

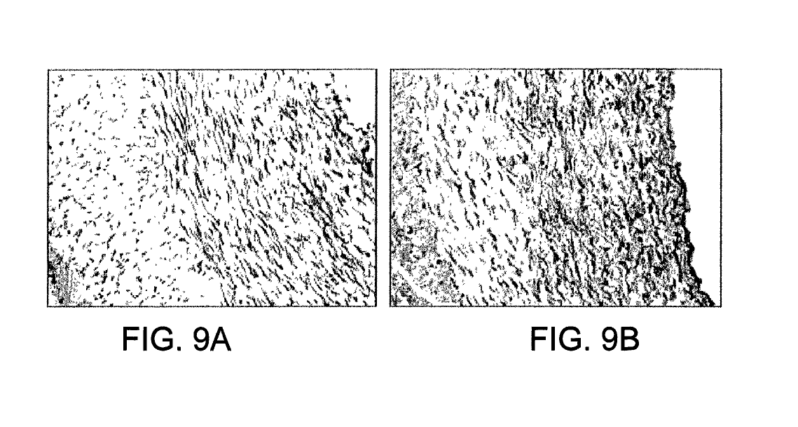

Confocal microscopy verified the expression of GFP and RFP in transferred cells by detecting their emitted fluorescence. The presence of either GFP or UP enhanced the cytoplasm of VSMCs, whose fibrous morphology was clearly delineated (FIG. 8b&d). In contrast, only minimal autofluorescence was detected in the untransferred tissue (FIG. 8a&c). Average signal intensities were measured by confocal microscope for untransferred and transferred arterial tissue for all five cases and compared using t-tests (Intercooled Stata 6.0; Stat Corp, TX) (TABLE 1). The fluorescence emitted by GFP-transferred tissue exhibited significantly higher signal intensities than autofluorescence in untransferred tissue, and similarly for RFP-transferred tissue. These results were corroborated by immunohistochemistry, which confirmed the success of the gene transfer and fluorophore expression. Staining for GFP showed the presence of the protein in the wall of the transferred artery, which appeared to have brown precipitates from the formation of the antibody-GFP complexes (FIG. 9 A&B).

TABLE 1
Student t-test Results of Signal Intensities of Arterial Tissue
Specimens Measured by Confocal Microscopy.

| | Control Tissue (AU) | Transferred Tissue (AU) |
|---|---|---|
| GFP (n_{obs} = 81) | 44.7 ± 17.4 | 50.7 ± 12.6 |
| RFP (n_{obs} = 54) | 57.8 ± 10.7 | 66.6 ± 11.5 |

Note: p < 0.05 for both GFP and RFP. Number of observations (n_{obs}) reflects the combined 27 measurements of GFP (n = 3) and RFP-transferred (n = 2) arteries and their respective controls. Data expressed as mean ± SD. AU is absolute units of signal intensity.

The results shown in Table 1 revealed statistically significant differences (p<0.05) between control and target tissues for both GFP and RFP trials. Moreover, results of the immunohistochemical staining for GFP in FIG. 9 indicated the presence of GFP in the arterial wall. Hence, the corroborative results from confocal microscopy and immunohistochemistry signified that the SMCs were expressing fluorophores which emitted fluorescence detected by optical imaging.

The experiments to detect GFP- and RFP-fluorescence in vivo by directly contacting the vessel wall. The results obtained from this method illustrate that the imaging method used herein, can discern the difference between normal and transfected tissue. Furthermore, the images show the areas where the fluorophores are located along the vessel wall. Thus, the optical imaging method is able to detect gene expression and map protein localization upon direct contact with the artery.

Exogenous fluorophores such as GFP and RFP enable the detection of gene expression or proteins by optical imaging. The results show that our optical imaging system can differentiate between untransferred and transferred tissue as well as map areas of gene expression.

### EXAMPLE 2

### Optical Imaging of Green Fluorescent Protein Markers for Tracking Vascular Gene Expression: A feasibility study in human tissue-like phantoms

### Human tissue-like Phantom

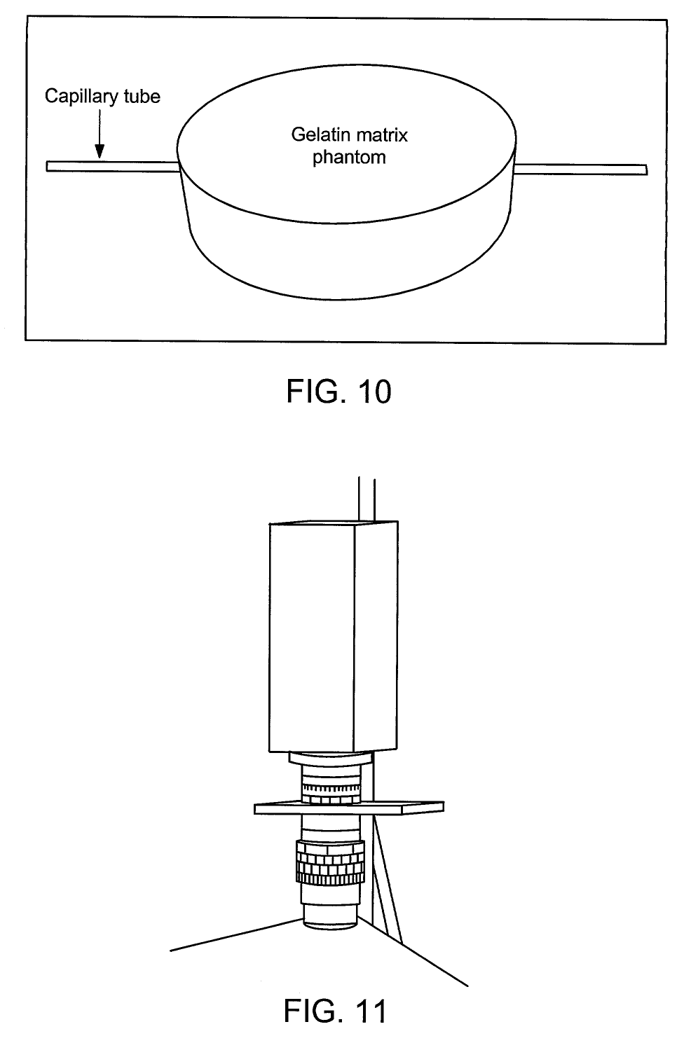

A set of gelatin matrix phantoms simulating human tissue-like properties were developed based on the model proposed by Durkin et al. In the phantoms, polystyrene microspheres were used as scatterers and human hemoglobin as absorbers. The human hemoglobin was purchased from Sigma Chemical Co. (St. Louis, Mo.) and was used at 2% by volume red blood cells (RBCs) in concentration. Polystyrene microspheres at 1.05-tun diameter were purchased from Polysciences Inc. (Warrington, Pa.), and were concentrated at 0.625%-by volume in the sample solution. The phantom matrix was constructed with Knox unflavored gelatin in PBS solution. The fluorophores FAD and Rodamine B were not used in the sample due to possible interference with GFP fluorescence. A capillary tube was selected to simulate the arterial geometry containing the transfected fluorescent proteins in vascular gene transfection. As illustrated in FIG. 10, the capillary tube made from glass with an inner diameter of 1.20 mm and a wall thickness of 0.40 mm, was embedded inside the petri dishes filled with gelatin matrix at 1, 2, 3, 5, and 7 mm depths. We first filled the tubes with PBS solution (as a control), and then imaged the PBS-containing tube using the optical imaging system. Subsequently, we injected the GFP-transfected smooth muscle cells (SMC) suspension (8 million cells/mL) into the same tube to replace the PBS. Then, we performed optical imaging to detect GFP emission from SMCs. Two concentrated GFP/SMC solutions at 30% and 15% of transfected cells/volume were injected to correlate with the detected fluorescent signal intensity.

### Optical Imaging System

The optical imaging system comprised a cooled charge-coupled device (CCD) camera (Sensi-Cam QE, Cooke Corp., MI) that interfaced to an image-grabber installed on a personal computer. A fiber-optic ring light (Nevoscope, Translite, TX) was connected to a broadband 250 W halogen light source (Schott Group, Germany). A series of lenses (Fujinon-TV, Fuji Photo Optical, Japan; Nikon AF Nikkor, Japan) along with the emission filter on a slider were mounted on the CCD camera.

The excitation filter (XF 1072, Omega Optical Inc., VT) was placed inside the light source to pass only the excitation band (460 nm-490 nm) that is specific to GFP. The emission filter (XF 3080, Omega Optical Inc., VT) passed the narrow band (500 nm-530 nm) that encompassed the emission wavelength of GFP. The excitation light photons irradiated on the surface of medium went through multiple scattering and absorption, and then reached the fluorophore layer. The absorbed photons at the fluorophore layer were remitted back at the emission wavelength. The collection geometry for signal detection was through an aperture of 1.20 cm diameter situated within the ring light.

FIG. 11 presents the experimental set-up; the entire imaging assembly was supported by an optical stand and the fiber optic ring light was placed on the surface of the gelatin matrix phantom (FIG. 10). The elevation of the imaging system was carefully adjusted to gently place the system on the surface of the phantom. Images were collected at various exposure times of 1-6 seconds to allow sufficient exposure with excitation light. IPLab Scientific Image processing v 3.070 (Scanalytics Inc., VA) software was used to perform the image intensity measurements. The results were analyzed with the student's t-test.

### Monte Carlo Simulation

Monte Carlo simulations of the phantom experiment were carried out, to validate and improve the performance of the optical imaging system. The Monte Carlo simulations refer to simulations of physical processes based on a stochastic model. In this particular study, the propagation of excitation light was first simulated at some wavelength, λₓ into the tissue model by a Monte Carlo program and then modeled the remitted photon propagation to the top surface of the medium at the emission wavelength, λₘ where λₘ≧λₓ, with a separate Monte Carlo program.

A program called MCML was written in standard C language for Monte Carlo simulation of photon propagation in multi-layered tissues, which dealt with only responses to an infinitely narrow photon beam normally incident on the surface of the tissue. However, all light source beam profiles had finite size beam geometry. Through a convolution process over the impulse response given by the output of MCML, the response to finite size beam geometry was computed. CONV was the computer program implementation of the convolution process response to Gaussian beams and circularly flat beams.

The imaging device used had a ring light source, which had neither a Gaussian nor a circularly flat beam profile. By utilizing the linearity and spatial invariance properties of convolution, the ring light geometry was modeled by subtracting the convolution response of a narrower beam from the response of a wider beam. By looking at the radial fluence distribution profile and the intensity profile of actual ring light illumination, the wider beam was selected to be a Gaussian beam with the outer diameter (1.70 cm) of the ring light. The narrow beam was selected to be a flat beam of 1.0 cm diameter, which was smaller than the inner diameter (1.56 cm) of the ring light source. The presence of an edge effect due to radial diffusion in the circularly flat geometry was taken into consideration in selecting the width of the narrow flat beam. The excitation light irradiance rate was measured by a power meter and found to be 40 mW/cm², which was used in the convolution process. After modeling the ring light beam response, illumination efficiencies of a ring beam, a Gaussian beam, and a circularly flat beam, were compared by simulating the fluence rate as a function of depth. The Gaussian beam 1/e² diameter and the circularly flat beam diameter were selected to be 1.70 cm for the convolution process.

The MCML program was modified to simulate isotropic propagation such that a semi-infinite, homogeneous, single layer model with a layer of isotropic fluorescent point sources at particular depths was simulated for the phantom model. Autofluorescence was not considered in the simulations, as it was not in the phantom model. The optical properties of the phantom used in the simulations are compiled below in Table 2. The refractive index of the gelatin was assumed to be 1.37 and the ambient medium above and below the layer was assumed to be 1.0.

TABLE 2
Optical properties of the phantom at
excitation and emission wavelengths.

| | GFP |
|---|---|
| Quantum Yield | 0.60 |
| Excitation wavelength (nm) | 489 |
| μₐ (cm⁻¹) | 1.25 |
| μ_{g} (cm⁻¹) | 275 |
| g | 0.942 |
| Emission wavelength (nm) | 508 |
| μ_{af} (cm⁻¹) | 1.25 |
| μ_{gf} (cm⁻¹) | 262.5 |
| g_{f} | 0.942 |

The average fluorescence signal intensity of 30% GFP (1024 a.u.) was significantly different (p<0.05) from the PBS (649 a.u.). There was a significant difference (p<0.05) in the signal intensity between 30%GFP (1108 a.u.) and 15% GFP (622 a.u.). No significant difference in signal intensity was observed between 15% GFP (611 a.u.) and PBS (701 a.u.) (Table 3).

TABLE 3
Average fluorescence signal intensity
(a)

| | | |
|---|---|---|
| 30% GFP | PBS | t-statistics |
| 1024 a.u. | 649 a.u. | p = 0.004 < 0.05 |

(b)

| | | |
|---|---|---|
| 30% GFP | 15% GFP | t-statistics |
| 1108 a.u. | 622 a.u. | p = 0.004 < 0.05 |

(c)

| | | |
|---|---|---|
| 15% GFP | PBS | t-statistics |
| 611 a.u. | 701 a.u. | p = 0.2 > 0.05 |

FIG. 12 displays the fluorescence detected from the 30% GFP sample and the lack of signal in the control PBS. The remitted fluorescence from the 5 mm thick phantom was captured with 6-second signal integration with the CCD camera. There was no detectable signal intensity difference between the 15% GFP and PBS. The simulated ring light beam response with a convolution process was used to obtain the remitted fluorescence as a function of radial distance at 5 mm depth. The remitted fluorescence at 5 mm depth was computed by multiplying the fluence rate by fluorescence concentration and the quantum yield of GFP.

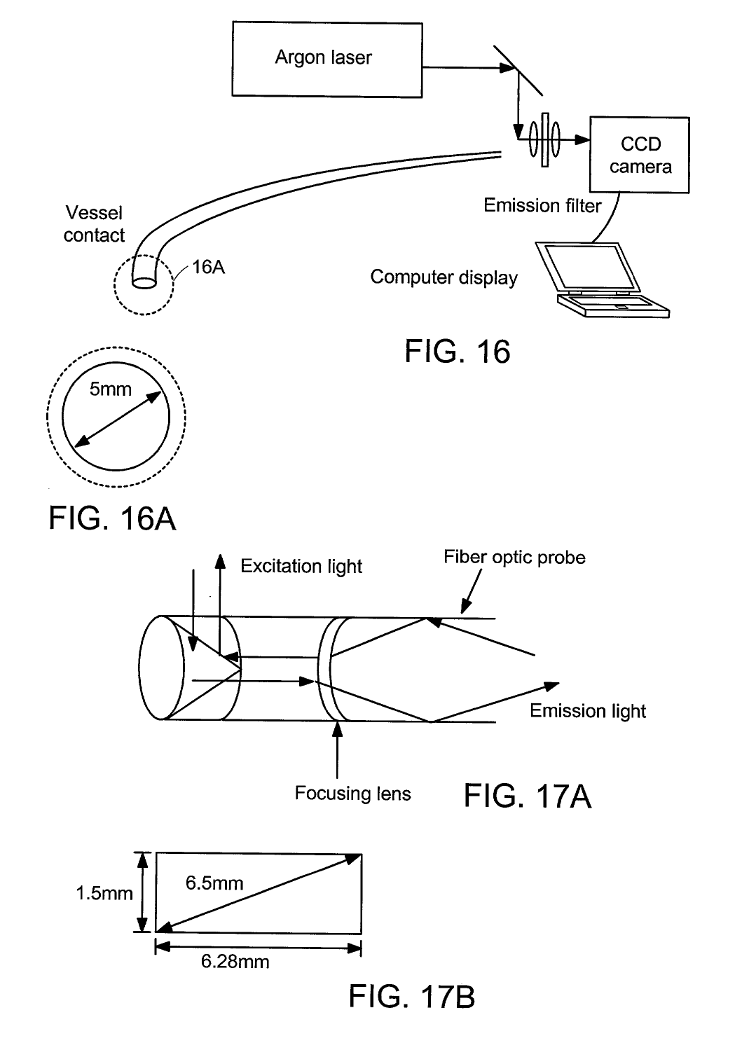

The maximum remitted fluorescence rate was about 0.2 mW/cm², positioned between radial distances 0.6 cm and 0.8 cm away from the center of the beam. This result coincides with fluorescence intensity images collected with the CCD camera (FIG. 12A). The signal void in the center portion of image displayed on FIG. 12A was due to the lack of excitation and fluorescence remittance near the central axis of the ring light beam. FIG. 16 shows a fourth-order polynomial line fit over the convolved response, giving an overall beam profile of the ring light illumination geometry.

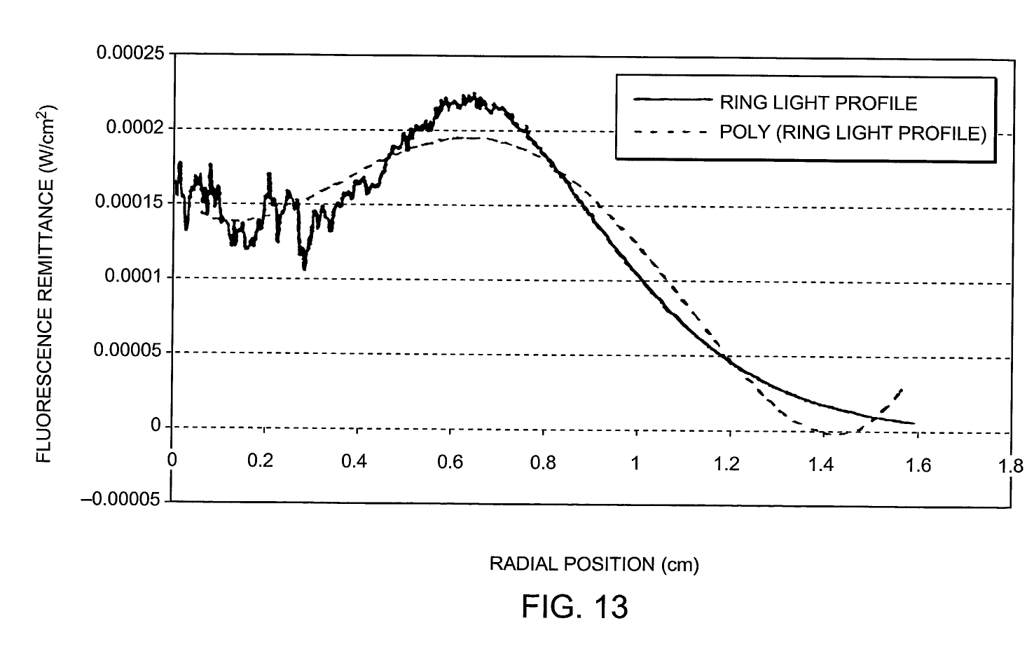

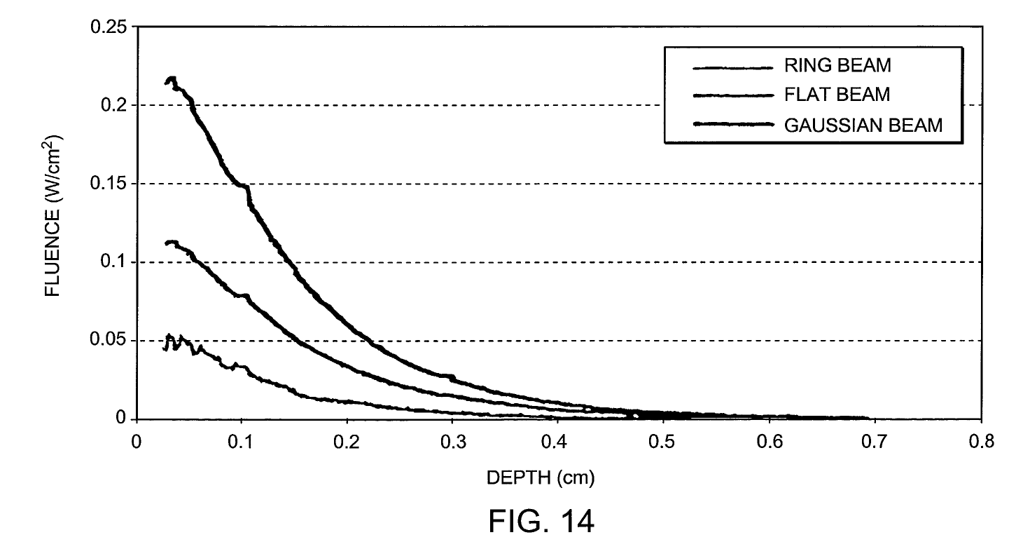

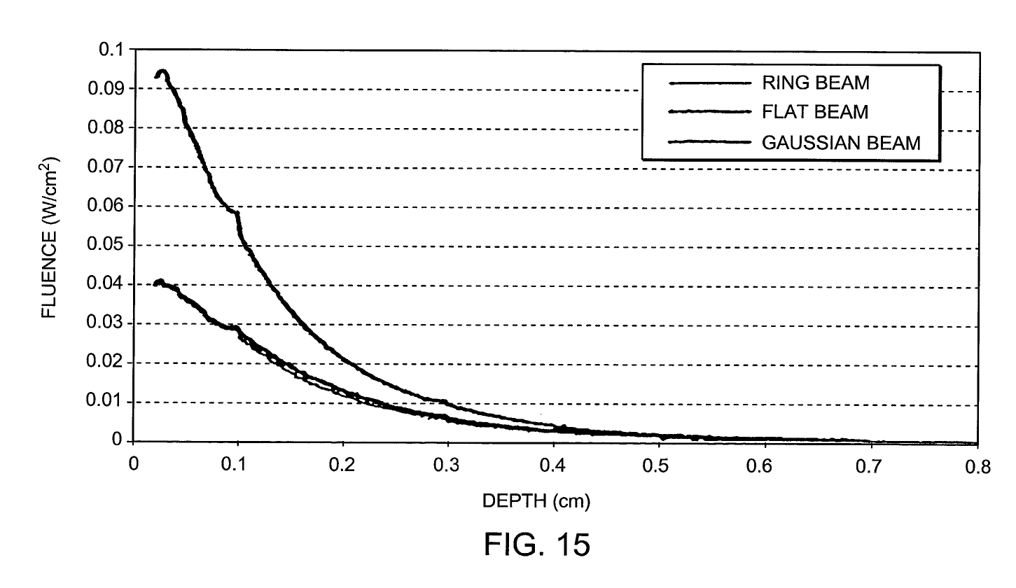

Remitted fluorescences as functions of radial distance were computed for a ring beam, a Gaussian, and a flat beam profiles at the central axis of the beams, shown in FIG. 17, and 0.8 cm away from the central axis (FIG. 13). At 1 mm depth, a Gaussian beam produced a fluence rate of 154 mW/cm², a flat beam produced a fluence rate of 81 mW/cm²; and a ring beam produced a fluence rate of 35 mW/cm².

Back-scattered light augmented the incident beam, yielding an internal fluence rate that exceeded the irradiance delivered at the surface. As photons penetrated deeper into the medium, the fluence rates from all three beams decreased in an exponential fashion. At the central axis and 5-mm deep into the tissue, the Gaussian beam fluence rate reduced to 5 mW/cm², the flat beam fluence rate to 3 mW/cm², and the ring beam decreased to 1 mW/cm².

FIG. 13 shows the fluence distributions as functions of depth at a radial distance of 0.8 cm, approximately at the radius of the beam geometries. Unlike at the central axis, the flat beam produced a higher fluence rate than a Gaussian or a ring beam at the beam edge. At 1-mm depth around the radial edge of beams, the flat beam produced a fluence rate of 59 mW/cm², Gaussian beam produced a fluence rate of 29 mW/cm², and the ring beam produced a fluence rate of 28 mW/cm². At 5-mm depth, the flat beam fluence rate was 2 mW/cm², and the Gaussian and ring light beams produced approximately 1 mW/cm² fluence rates. The fluence rate distributions of Gaussian and ring beams were very close to each other around the beam radius.

The experimental results and the Monte Carlo simulations showed that GFP signal was detected up to 5-mm depth in a human tissue-like phantom. A comparison of simulation results of fluence rate and distributions of the three beam geometries, points to the flat beam geometry for the most efficient excitation light delivery. Flat beam produces more than a two-fold increase in fluence rate compared to a ring beam at the center of the beam and at the periphery. Fluorescent markers with longer excitation/emission wavelengths and higher quantum yield will enhance the non-invasive detection efficiency.

### EXAMPLE 3

### Design of Intravascular and Minimally Invasive Optical Imaging Probes to Track Vascular Gene Expression—A Monte Carlo Simulation Analysis

### Methods

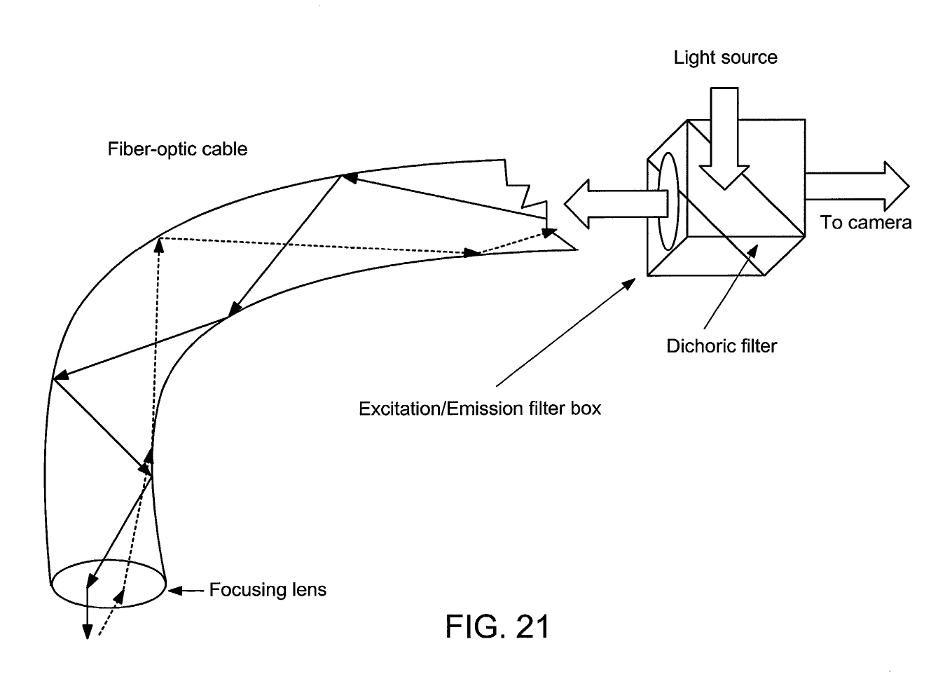

The intravascular catheter probes (FIG. 17) were designed for intravascular optical imaging of deep-seated arteries. The minimally invasive optical source-detector probes (FIG. 21) considered in the analysis were designed for percutaneous direct contact on the vessel surface.

The intravasuclar probe is 2 mm in diameter and possesses a modified cylindrical source-detector portion from the percutaneous probe shown in FIG. 17. The intravascular probe head contact area on the vessel wall is shown in FIG. 17. The fluence distribution was set at the central axis with a 1.5 mm diameter Gaussian beam and represented a valid comparison to the excitation response of the described cylindrical beam head of the intravascular probe at a certain radial position. The incident beam power was selected to be 1 W/cm².

Light propagation in the vasculature was simulated by Monte Carlo modeling, in which individual packets of photons are followed as they pass through multiple scattering and absorption events. A program called MCML was written in standard C language for Monte Carlo simulation response to a point source normally incident on a tissue medium. One million photons were used to characterize impulse response in the aortic vessel wall. Fluence distribution at the GFP excitation wavelength (488 nm) was simulated for the intravascular probe on normal as well as on vessel walls with plaques. The optical properties of the aorta and fibrous plaque are presented in Table 4.

TABLE 4
Optical properties of tissues considered in the simulations.

| | | ^{a} | ^{s} | Thickness |
|---|---|---|---|---|
| Tissue | (nm) | (cm⁻¹) | (cm⁻¹) | ( m) |
| Intima | 476 | 14.8 | 237 | 286 ± 78 |
| Media | 476 | 7.3 | 410 | 872 ± 225 |
| Adventitia | 476 | 18.1 | 267 | 175 ± 114 |
| Plaque | 488 | 16.6 | 19 | 1130 |

For the percutaneous probe (FIG. 21), a circularly flat beam with a diameter of 5 mm was selected for the excitation beam as it produced high fluence distribution in the periphery of the fiber. The program CONV, was used to produce the convolution response of the finite size source beam responses. The incident beam power was 1 W/cm². Simulations were performed to analyze the excitation light fluence distribution in the aortic wall.

The aorta consists of three layers: a) the intima, which is a thin endothelial lining on the inside of the vessel; b) the media, a thick layer of dense layers of collagen, elastin and smooth muscle cells; and c) the adventitia, the outside coating of randomly arranged thick collagen fibers. In the simulations, consideration was for a 1 mm thick fibrous plaque layer on the intima.

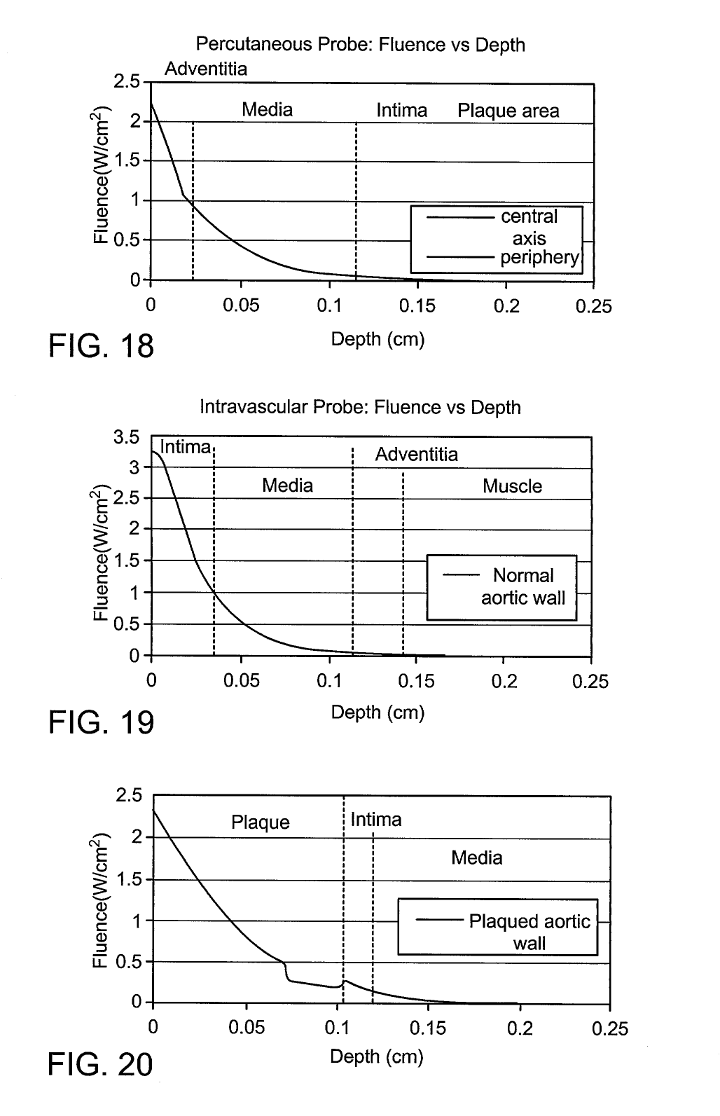

The fluence distribution of the percutaneous probe at the central axis and at the edge of the excitation probe was simulated and plotted in FIG. 18. The closeness of the fluence distribution functions at the two locations revealed the uniform excitation of the probe over the encompassing area. The media-intima interface area, where most of the GFP were located had an average fluence distribution of 0.12 W/cm².

The fluence distribution functions of the intravascular catheter probe on normal aorta and on aorta with plaques were presented in FIGS. 19 and 20. In the normal wall, a 0.06 mm thick area around the media-intima interface had a fluence rate of 0.7 W/cm². The slope of the fluence distribution function in the fibrous plaque was smaller compared to that of the intima in the normal aorta. The average fluence distribution in the 0.06 mm thick area around the media-intima interface was 0.1 W/cm².

The average fluence distribution in the 0.06 mm thick area encompassing the media-intima interface increased seven fold in the case of the intravascular probe used on a normal aortic wall. In the diseased aortic wall, the fluence distribution remained comparable to the fluence distribution of the percutaneous probe.

Atherosclerotic plaques can be classified basically into three types: fibrous plaque, lipid plaque, and calcified plaque. This example simulated the diseased artery with fibrous plaque. The refraction index mismatch at the interface of the fibrous plaque and the intima increased the excitation light intensity losses due to scattering (see FIG. 20). The region around the media-intima interface, where most of the transfected GFP would be found, is also an area where high scattering of light occurs by the Rayleigh process. The striated bands of collagen fibrils with small fractions of the excitation wavelength periodicity could cause scattering in the media.

The cylindrical contact surface of the source-detector head of the intravascular probe is suited for optimal power delivery without causing heating of the vasculature. The increased contact surface area spread the power delivered via optical fiber uniformly around the vessel surface.

The optimal probe design for minimally invasive tracking of gene expression does not depend only on the efficient light delivery for excitation. The fluorescence quantum yield, fluorescent marker transfection rate, the path length of fluorescent light in the tissue, and the tissue absorption coefficient for the fluorescence are other significant factors that determine the successful detection of fluorescence by the probe. The fluence distribution produced by the intravascular probe comparable to that of the percutaneous probe provides a way of monitoring gene expression in the deep-seated arteries. The simulation studies provided valuable information about the design of optical probes for tracking gene expression in both superficial and the deep-seated vasculature.

The foregoing description of the invention is merely illustrative thereof, and it is understood that variations and modifications can be made without departing from the spirit or scope of the invention as set forth in the following claims.

What is claimed:

1. A method for detecting intravascularly administered molecules, comprising:
administering to a patient a composition having a detectable component; and
detecting the administered composition with use of a digital optical imaging system.

2. The method of claim 1 wherein the composition is administered intravascularly to the patient.

3. The method of claim 1 or 2 wherein after administration the composition is excited with activating radiation to thereby produce a fluorescent signal.

4. The method of any one of claims 1 through 3 wherein the imaging system comprises an optical component.

5. The method of any one of claims 1 through 4 wherein the imaging system comprises a charge coupled device detector which detects a fluorescent signal emitted from the administered composition.

6. The method of any one of claims 1 through 5 wherein the imaging system comprises a central processing unit.

7. The method of claim 6 wherein the central processing unit manipulates data received regarding fluorescent signal emitted from the administered composition.

8. The method of any one of claims 1 through 7 wherein the digital imaging system comprises one or more of a central processing unit, a digital angioscope, a digital optical camera, an irrigation system, and an apparatus for intravascular administration of the composition.

9. The method of claim 8 wherein the central processing unit is a computerized control and/or display system.

10. The method of claim 8 wherein the central processing unit is a computer with programmable software that increases magnification of an image, analyzes the image, stores the image, and/or displays the image.

11. The method of claim 8 wherein the digital angioscope is controlled by the central processing unit.

12. The method of claim 8 wherein one end of said digital angioscope is connected to the photosensitive charge-coupled device and a tip end which is inserted into an intravascular location.

13. The method of claim 8 wherein the digital angioscope transfers radiation from a radiation source to the location of a molecule with a fluorescent component, thereby exciting the molecules and detecting the fluorescent signals emitted from the location of a molecule with a fluorescent component.

14. The method of claim 8 wherein the digital angioscope transfers light from a light source to excite a molecule with a fluorescent component and detects auto fluorescent signals emitted from cells in a target vessel wall.

15. The method of claim 8 wherein the digital angioscope comprises an optical rotating probe.

16. The method of claim 15 wherein the optical rotating probe is positioning at a distal end of a digital angioscope.

17. The method of claim 15 wherein the optical rotating probe is configured to fit within an anatomic channel or cavity of the patient.

18. The method of claim 15 wherein said optical rotating probe is used in combination with balloon angioplasty, stent placement, or angioscopic examination.

19. The method of claim 15 wherein the optical rotating probe is positioned within a targeted vessel under guidance of an imaging modality.

20. The method of claim 19 wherein the optical rotating probe comprises a tip coated with alloy markers to enable the visualization during positioning of the probe within a patient.

21. The method of claim 15 wherein the optical rotating probe transmits excitation light into the target to excite the fluorescent composition.

22. The method of claim 15 wherein the optical rotating probe collects fluorescent/luminescent lights emitted from a transgene-target, transfer the light signals to a CCD camera, and then routes them into the computer.

23. A method for detecting intravascularly administered molecules, the method comprising:
providing a digital optical imaging system;
administering to a patient a composition having a detectable component; and detecting the administered composition with the imaging system;
wherein the composition is excited by an external radiation source and thereby emits a detection signal.

24. The method of claim 23 wherein the composition emits a fluorescent signal.

25. The method of claim 23 or 24 wherein the detection signal is inputted to a charge coupled device detector.

26. The method of claim 25 wherein the charge coupled device routes the input to a central processing unit.

27. A system for intravascularly tracking vascular gene therapeutic procedures and imaging of internal elastic lamina (IEL) of deep-seated vessels, including arteries and veins, the system comprising:
a computer;
a digital angioscope comprising an optical rotating probe for generating vessel wall images at a 360° view; wherein,
the angioscope operated under the control of a computer, the angioscope either transfers activating radiation to excite extrinsic fluorescence or intrinsic fluorescence emitted from a target vessel wall; wherein a digital optical camera detects the fluorescent signals and is connected between the computer and the angioscope.

28. A system for intravascularly tracking vascular gene therapeutic procedures, comprising:
a computer and a digital angioscope for detecting extrinsic fluorescence or intrinsic fluorescence emitted from a target vessel wall of a patient.

29. The system of claim 27 or 28 wherein the system additionally comprises an irrigation system.

30. The system of claim 29 wherein the irrigation system comprises an irrigating pump, a working channel connected on one end to the pump and a distal end positioned proximate to an optical fiber of the digital angioscope.

31. The system of claim 30 wherein the irrigation system comprise an irrigant.

32. The system of claim 31 wherein the irrigant is saline or carbon dioxide gas.

33. The system of any one of claim 30 through 32 wherein the system comprises one or more of an external light source; a fiberoptic light guide; and one or more wavelength selective optical lens.

34. The system of claim 33 wherein the external light source is a halogen lamp.

35. The system of claim 33 wherein the external light source is a laser beam or an infrared beam.

36. The system of claim 33 wherein the external light source emits light is selected according to the optical imaging marker used.

36. The system of claim 33 wherein the fiberoptic light guide guides photons having wavelengths selected according to the optical imaging marker used.

37. The system of claim 33 wherein the fiberoptic light guide is flexible.

38. The system of claim 33, wherein the fiberoptic light guide is configured to fit into a blood vessel.

39. The system of claim 33 wherein the one or more wavelength selective optical lenses determine the wavelength of light passing through the lenses.

40. The system of claim 39 wherein the wavelength selective optical lenses are selective for light with wavelengths according to the optical imaging marker used.

41. A method for detecting intravascularly administered molecules, comprising:
administering to a patient a composition having a detectable component;
detecting the administered composition with use of a digital imaging system and a probe external to the patient.

42. The method of claim 41 wherein the probe is in contact with the patient's skin during detection.

43. The method of claim 41 or 42 wherein the composition is administered intravascularly to the patient.

44. The method of any one of claims 41 through 43 wherein the imaging system comprises a central processing unit and an external digital optical camera.

45. The method of claim 44 wherein the central processing unit comprises a computerized control and display system.

46. The method of claim 44 or 45 wherein the external digital optical camera comprises a hand-held probe operated under the control of the central processing unit.

47. The method of any one of claims 44 through 46 wherein the digital camera comprises a spatially coherent fiber optic bundle.

48. The method of any one of claims 44 through 47 wherein the digital camera can transfers external light to excite the extrinsic fluorescence or autofluorescence within a vessel and detects fluorescent signals emitted from the vessel wall.

49. The method of claim 48 wherein the digital camera generates vessel wall images in a cross-sectional view, and is connected between a central processing unit and an external light source.

50. A method for detecting intravascularly administered molecules, comprising:
administering to a patient a composition having a detectable component;
detecting the administered composition with use of a digital imaging system and a probe transpercutaneously inserted within the patient.

51. The method of claim 50 wherein the probe is a fiberoptic probe.

52. The method of claim 50 and 51 wherein the probe is positioned proximate to a transgene-targeted vessel of the patient.

53. The method of any one of claims 1 through 26 or 41 through 52 wherein the detectable composition comprises a nucleic acid compound.

54. The method of claim 53 wherein the detectable composition can express a polypeptide upon administration.

55. The method of any one of claims 1 through 26 or 41 through 54 wherein the administered composition comprises a fluorescent molecule selected from the group consisting of green, red, blue, green, cyan, and yellow.

56. The method of any one of claims 1 through 26 or 41 through 54 wherein green fluorescent protein is a detected marker.

57. The method of any one of claims 1 through 26 or 41 through 54 wherein enhanced green fluorescent protein is a detected marker.

58. The method of any one of claims 1 through 26 or 41 through 54 wherein red fluorescent protein is the optical marker.

59. The method of any one of claims 1 through 26 or 41 through 54 wherein the detectable composition comprises a labeled molecule that is a vector, antibody, drug, biomarker, nucleic acid compound, protein, polypeptide, amino acid, or fragment thereof.

60. The method of any one of claims 41 through 49 wherein the imaging system can detect fluorescence up to about 5 mm in depth in the skin of the patient.

61. The method of any one of claims 1 through 26 or 41 through 60 wherein the light source is a ring beam, a Gaussian beam or a flat beam.

62. The method of claim 61 wherein the light source used in producing fluorescence is a flat beam.

63. The method of any one of claims 50 through 54 wherein a fiberoptic probe is dimensioned to fit in any transpercutaneous interventinal device.

64. The method of claim 63 wherein the interventinal device functions to provide transpercutaneous biopsy and draining.

65. The method of any one of claims 50 through 54, 63 or 64 wherein a fiberoptic probe is inserted nearby a transgene-targeted vessel via minimally surgical incision of the skin of the patient.

66. The method of claim 65 wherein the fiberoptic probe is positioned with the body proximate to a transgene-targeted vessel under guidance of an imaging modality.

67. The method of claim 50 through 54 or 63 through 66 wherein the fiberoptic probe is coated with alloy markers to enable the visualization of the probe during positioning under an imaging modality.

68. The method of claim 1 through 26 or 41 through 67 wherein a fiberoptic probe transmits the excitation light into the target to excite the fluorescent/luminescent molecules.

69. The method of claim 68 wherein the fiberoptic probe collects the fluorescent/luminescent lights emitted from the target, transfer the light signals to a computer.

70. The method of any one of claims 1 through 26 or 41 through 69 wherein a vascular gene therapy procedure is detected.

71. The method of any one of claims 1 through 26 or 41 through 69 fluorescence expressing molecules are detected in the organ of the patient.

72. The method of any one of claims 1 through 26 or 41 through 69 wherein antigens diagnostic of a disease or disorder is detected.

73. The method of any one of claims 1 through 26 or 41 through 69 antigens from different stages of disease progression are detected.

74. The method of any one of claims 1 through 26 or 41 through 69 wherein two or more optical imaging systems are used to detect the localization of fluorescence expressing molecules administered for diagnosis, drug delivery or gene therapy to a patient.

75. The method of claim 74 wherein two or three optical imaging systems are employed.

76. Use of a method or system of any one of claims 1 through 75 to detect and assess a vascular gene therapy procedure.

77. Use of a method or system of any one of claims 1 through 75 fluorescence expressing molecules are detected in the organ of the patient.

78. Use of a method or system of any one of claims 1 through 75 to detect an antigen diagnostic of a disorder or disease.

79. Use of a method or system of any one of claims 1 through 75 for the diagnosis or treatment of atherosclerosis.
